(19) United States
(12) Patent Application Publication
(10) Pub. No.: US 2024/0271094 A1
(43) Pub. Date: Aug. 15, 2024

(54) COMPOSITIONS AND METHODS FOR PATHOGEN INACTIVATION OF PLATELETS

(71) Applicant: Cerus Corporation, Concord, CA (US)

(72) Inventors: William GREENMAN, Concord, CA (US); Adonis STASSINOPOULOS, Del Mar, CA (US); Elan WEINER, Concord, CA (US); Peter BRINGMANN, Concord, CA (US); Felicia SANTA MARIA, Bay Point, CA (US)

(21) Appl. No.: 18/416,698

(22) Filed: Jan. 18, 2024

Related U.S. Application Data
(63) Continuation of application No. 16/137,391, filed on Sep. 20, 2018, now abandoned.
(60) Provisional application No. 62/616,338, filed on Jan. 11, 2018.
(60) Provisional application No. 62/586,739, filed on Nov. 15, 2017.
(60) Provisional application No. 62/561,157, filed on Sep. 20, 2017.

(51) Int. Cl. C12N 5/078; A01N 1/02; A61K 35/19; A61K 41/17; A61L 2/00; A61M 1/02; A61M 1/36

(52) U.S. Cl. CPC C12N 5/0644; A01N 1/0215; A01N 1/0294; A61K 41/17; A61L 2/0029; A61L 2/0047; A61M 1/0272; A61K 35/19; A61M 1/3683; A61M 2202/0427; C12N 2501/999; C12N 2529/10

(57) ABSTRACT

Provided are methods, kits, and compositions for preparing platelet compositions suitable for infusion, including improved methods, compositions, and kits for pathogen inactivation of an apheresis-derived preparation of platelets.

### CROSS-REFERENCE TO RELATED APPLICATIONS

This application claims priority to U.S. Provisional Patent Application No. 62/616,338, filed Jan. 11, 2018, U.S. Provisional Patent Application No. 62/586,739, filed Nov. 15, 2017, and U.S. Provisional Patent Application No. 62/561,157, filed Sep. 20, 2017, the disclosures of each of which are incorporated herein by reference in their entirety.

### TECHNICAL FIELD

The present disclosure provides methods, kits, and compositions for preparing platelet compositions suitable for infusion. In some aspects, the disclosure provides improved methods, kits, and compositions for pathogen inactivation of a preparation of platelets, including an apheresis-derived preparation of platelets.

### BACKGROUND

Blood component collection and processing serves a critical role in healthcare worldwide, and millions of units of donated blood components are collected by blood banks each year. While some units of whole blood are collected from donors and used directly for transfusion, most donations are generally separated into the blood components (red blood cells, platelets, and plasma) for more specific therapeutic use. Separation may be either following collection of whole blood donations or at the point of collection if using a suitable separation device system, such as an apheresis collection device. Individual blood components are then used in treating different medical needs and conditions based on therapeutic need.

Platelets play a key role in hemostasis, clot stability and retraction, as well as in vascular repair and anti-microbial host defense. A variety of methods are used to collect and store platelet blood products for clinical use. Collection of platelets from whole blood donations is generally in the form of platelet concentrates (PC), obtained using processing methods such as a buffy coat or platelet rich plasma method. Platelets are also obtained from apheresis collection, which utilizes an automated device that separates donor platelets from donor blood and returns remaining blood components (e.g., red blood cells and plasma) to the donor during the donation process.

To minimize the risk of infecting an individual receiving a blood product, it is important to ensure that blood products, such as platelets, be free of pathogens. Testing for the presence of a blood pathogen is limited by the pathogens tested for and assay sensitivity. As an alternative or supplement to testing for pathogens, methods are known in the art for inactivating pathogens using various compound (e.g., chemical, photochemical)-based inactivation methods (e.g., as disclosed in Schlenke et al., Transfus Med Hemother, 2014, 41, 309-325 and Prowse, Vox Sanguinis, 2013, 104, 183-199). Such inactivation methods may require specific guard band ranges for input platelet volumes and platelet numbers in order to achieve a desired compound concentration for pathogen inactivation. For example, a minimum concentration may be defined by the concentration necessary to achieve a certain level of pathogen inactivation and a maximum concentration may be defined by the concentration at which the treatment may have an impact on the function of the treated blood product. Donation volumes and platelet numbers can significantly vary from donor-to-donor or donation-to-donation, and to maximize use of pathogen inactivation systems for blood component donations an improved level of flexibility of processing remains desirable. Methods, kits, and compositions for achieving increased flexibility and improved productivity in processing are described herein.

All references cited herein, including patent applications and publications, are incorporated by reference in their entirety.

### BRIEF SUMMARY

In one aspect, provided is a method of preparing a platelet composition (e.g., pathogen inactivated platelet composition), comprising (a) providing a solution comprising a platelet additive solution (PAS) and a pathogen inactivation compound (PIC); (b) admixing the solution of step (a) with a preparation of platelets; and (c) subjecting the admixture of step (b) to light sufficient to photochemically inactivate a pathogen, if present, thereby yielding the platelet composition. In some embodiments, a method of preparing a platelet composition (e.g., pathogen inactivated platelet composition) is provided, comprising (a) providing in a first container a solution comprising a platelet additive solution (PAS) and a pathogen inactivation compound (PIC); (b) admixing the solution of step (a) with a preparation of platelets; and (c) subjecting the admixture of step (b) to light sufficient to photochemically inactivate a pathogen, if present, thereby yielding the platelet composition.

In some embodiments, providing in a first container a solution comprising a PAS and a PIC comprises first combining a solution of PAS and a solution of PIC to yield the solution comprising a PAS and a PIC. In some embodiments, the method comprises, prior to step (a), combining a solution of PAS and a solution of PIC to yield a solution comprising a PAS and a PIC. In some embodiments, the solution of PAS is from a PAS container (e.g., PAS storage container). In some embodiments, the solution of PIC is from a PIC container (e.g., PIC storage container). In some embodiments, the solution of PAS and solution of PIC are combined in the first container of step (a). In some embodiments, the first container of step (a) is the PAS container. In some embodiments, the first container of step (a) is the PIC container. In some embodiments, the solution of PAS and the solution of PIC are combined less than 24 hours (e.g., within 24 hours) before the admixing of step (b). In some embodiments, the admixing of step (b) occurs in the first container. In some embodiments, the admixing of step (b) occurs in a second container. In some embodiments, the admixing occurs in two or more second containers. In some embodiments, the preparation of platelets is prepared by an apheresis method. In some embodiments, the method further comprises, prior to step (b), connecting the first container to an apheresis device. In some embodiments, the PAS container is connected to an apheresis device. In some embodiments, the PIC container is connected to an apheresis device. In some embodiments, the second container is connected to an apheresis device. In some embodiments, the two or more second containers are connected to an apheresis device. In some embodiments, the preparation of platelets is prepared from one or more whole blood donation(s) by a buffy coat method or a platelet rich plasma (PRP) method. In some embodiments, the method further comprises, after step (c), transferring the platelet composition to a third container. In some embodiments, the method further comprises, after step (c), transferring the platelet composition to two or more third containers. In some embodiments, the third container comprises a compound adsorption device (CAD). In some embodiments, the third container is suitable for storage of the platelet composition. In some embodiments, the method further comprises, transferring the platelet composition from the third container to one or more fourth containers. In some embodiments, the one or more fourth containers is/are suitable for storage of the platelet composition.

In some embodiments, the solution of step (a) has a volume of between about 100 mL and about 1000 mL. In some embodiments, the solution of step (a) comprises the PIC at a concentration of about 15 μM to about 1500 μM. In some embodiments, the solution of step (a) comprises the PIC at a concentration of about 15 μM to about 235 μM. In some embodiments, the solution of step (a) comprises the PIC at a concentration of about 225 μM to about 235 PM. In some embodiments, the PIC is a psoralen. In some embodiments, the PIC is amotosalen. In some embodiments, the preparation of platelets comprises plasma, wherein the plasma comprises about 32 to 47% by volume of the admixture of step (b), with platelet additive solution (e.g., platelet additive solution with PIC) comprising the remaining volume. In some embodiments, the ratio of PAS to plasma by volume in the admixture of step (b) is about 65:35. In some embodiments, the total volume of the admixture of step (b) is about 100 mL to about 1000 mL. In some embodiments, the admixture of step (b) comprises the PIC at a concentration sufficient to result in inactivation of at least 1 log of a pathogen, if present. In some embodiments, the admixture of step (b) comprises the PIC at a concentration sufficient to result in inactivation of at least 3 logs of a pathogen, if present. In some embodiments, the admixture of step (b) comprises the PIC at a concentration sufficient to result in inactivation of at least 4 logs of a pathogen, if present. In some embodiments, the admixture of step (b) comprises the PIC at a concentration of about 5 μM to about 500 PM. In some embodiments, the admixture of step (b) comprises the PIC at a concentration of about 15 μM to about 150 μM. In some embodiments, the admixture of step (b) comprises the PIC at a concentration of about 15 μM to about 30 μM. In some embodiments, the admixture of step (b) comprises the PIC at a concentration of about 30 μM to about 150 μM. In some embodiments, the admixture of step (b) comprises the PIC at a concentration of about 30 μM to about 90 PM. In some embodiments, the admixture of step (b) comprises the PIC at a concentration of about 75 PM. In some embodiments, the admixture of step (b) comprises the PIC at a concentration of about 145 μM to about 155 μM. In some embodiments, the PAS comprises one or more of chloride, acetate, citrate, potassium, magnesium, phosphate, gluconate, glucose, and bicarbonate. In some embodiments, the method further comprises, prior to step (c), incubating the admixture of step (b) for a period of from 30 minutes to 24 hours. In some embodiments, the platelet composition comprises at least 2×10¹¹ platelets.

In some embodiments, the method is sufficient to inactivate at least 1 log of a pathogen, and wherein the platelet composition after step (c) is suitable for infusion into a subject without further processing to remove residual PIC or photoproducts thereof. In some embodiments, the method is sufficient to inactivate at least 4 log of a pathogen, and wherein the platelet composition after step (c) is suitable for infusion into a subject without further processing to remove residual PIC or photoproducts thereof. In some embodiments, the method is sufficient to inactivate at least 1 log of a pathogen, and wherein the platelet composition after step (c) is suitable for infusion into a subject without transferring the platelet composition to a container comprising a compound adsorption device (CAD). In some embodiments, the method is sufficient to inactivate at least 4 log of a pathogen, and wherein the platelet composition after step (c) is suitable for infusion into a subject without transferring the platelet composition to a container comprising a compound adsorption device (CAD). In some embodiments, the method is sufficient to inactivate at least 1 log of a pathogen, and wherein the platelet composition after step (c) comprises 5 μM or less of PIC. In some embodiments, the method is sufficient to inactivate at least 4 log of a pathogen, and wherein the platelet composition after step (c) comprises 5 μM or less of PIC. In some embodiments, the method is sufficient to inactivate at least 4 log of a pathogen, and wherein the platelet composition after step (c) comprises 2 μM or less (e.g., less than 2 μM) of PIC. In some embodiments, the method is sufficient to inactivate at least 4 log of a hepatitis E virus. In some embodiments, the platelet composition suitable for infusion into a subject comprises about 5 μM or less of PIC. In some embodiments, the platelet composition suitable for infusion into a subject comprises about 2 μM or less (e.g., less than 2 μM) of PIC. In some embodiments, the concentration of PIC in the admixture of step (b) is at least 10 μM. In some embodiments, the concentration of PIC in the admixture of step (b) is at least 30 μM.

In another aspect, provided is a kit for preparing a platelet composition, comprising (a) a first container comprising a solution comprising a platelet additive solution (PAS) and a pathogen inactivation compound (PIC), and (b) a second container suitable for containing a preparation of platelets in admixture with the solution comprising the PAS and the PIC, wherein the first container is not coupled to the second container.

In some embodiments, the first container is suitable for admixing the preparation of platelets with the solution comprising the PAS and the PIC. In some embodiments, the second container is suitable for admixing the preparation of platelets with the solution comprising the PAS and the PIC. In some embodiments, the second container is suitable for subjecting the preparation of platelets in admixture with the solution comprising the PAS and the PIC to light sufficient to photochemically inactivate a pathogen, if present. In some embodiments, the first container is suitable for subjecting the preparation of platelets in admixture with the solution comprising the PAS and the PIC to light sufficient to photochemically inactivate a pathogen, if present. In some embodiments, the second container comprises a compound adsorption device (CAD). In some embodiments, the second container is suitable for storing the platelet composition. In some embodiments, the kit further comprises a third container. In some embodiments, the third container comprises a compound adsorption device (CAD), and wherein the third container is coupled to the second container. In some embodiments, the kit further comprises at least one storage container, wherein the at least one storage container is suitable for storing the platelet composition, and wherein the at least one storage container is coupled to the second container or to the third container, if present. In some embodiments, the kit does not comprise a compound adsorption device (CAD).

In some embodiments, the solution comprising the PAS and the PIC has a volume of between about 100 mL and about 1000 mL. In some embodiments, the PIC is at a concentration of about 15 μM to about 1500 μM. In some embodiments, the PIC is at a concentration of about 225 μM to about 235 μM. In some embodiments, the PIC is a psoralen. In some embodiments, the PIC is amotosalen.

In some embodiments, the first container, the second container, or both the first container and second container is suitable for connecting to an apheresis device or to a container containing a preparation of platelets.

In another aspect, provided is a kit for preparing a platelet composition, comprising (a) a first container comprising a platelet additive solution (PAS); (b) a second container comprising a pathogen inactivation compound (PIC); and (c) a third container suitable for containing a preparation of platelets in admixture with the PAS and the PIC, wherein neither of the first and second containers is coupled to the third container. In some embodiments, the kit for preparing a platelet composition is a kit comprising (a) a first container comprising a platelet additive solution (PAS); (b) a second container comprising a pathogen inactivation compound (PIC); and (c) a third container suitable for containing a preparation of platelets in admixture with the with the PAS and the PIC, wherein the first and second containers are configured to be coupled to one another, and wherein neither of the first and second containers is coupled to the third container. In some embodiments, the kit for preparing a platelet composition is a kit comprising (a) a first container comprising a platelet additive solution (PAS); (b) a second container comprising a pathogen inactivation compound (PIC); and (c) a third container suitable for containing a preparation of platelets in admixture with the with the PAS and the PIC, wherein the first and second containers are coupled to one another, and wherein neither of the first and second containers is coupled to the third container. In some embodiments, the first and second containers are coupled to one another by a sealed but openable flow path (e.g., frangible member, frangible connector).

In some embodiments, the second container is suitable for combining the PAS with the PIC. In some embodiments, the second container is suitable for admixing the preparation of platelets with the PAS and the PIC. In some embodiments, the first container is suitable for combining the PAS with the PIC. In some embodiments, the first container is suitable for admixing the preparation of platelets with the PAS and the PIC. In some embodiments, the third container is suitable for admixing the preparation of platelets with the PAS and the PIC. In some embodiments, the third container is suitable for subjecting the preparation of platelets in admixture with the PAS and the PIC to light sufficient to photochemically inactivate a pathogen, if present. In some embodiments, the second container is suitable for subjecting the preparation of platelets in admixture with the PAS and the PIC to light sufficient to photochemically inactivate a pathogen, if present. In some embodiments, the first container is suitable for subjecting the preparation of platelets in admixture with the PAS and the PIC to light sufficient to photochemically inactivate a pathogen, if present. In some embodiments, the third container comprises a compound adsorption device (CAD). In some embodiments, the third container is suitable for storing the platelet composition. In some embodiments, the kit further comprises a fourth container. In some embodiments, the fourth container comprises a compound adsorption device (CAD), and wherein the fourth container is coupled to the third container. In some embodiments, the kit further comprises at least one storage container, wherein the at least one storage container is suitable for storing the platelet composition, and wherein the at least one storage container is coupled to the third container or to the fourth container, if present. In some embodiments, the PIC is a psoralen. In some embodiments, the PIC is amotosalen. In some embodiments, the first container, the second container, or both the first container and second container is suitable for connecting to an apheresis device or to a container containing a preparation of platelets. In some embodiments, the third container is suitable for connecting to an apheresis device or to a container containing a preparation of platelets. In some embodiments, the kit does not comprise a compound adsorption device (CAD).

In another aspect, provided is a composition comprising a pathogen inactivation compound (PIC) and a platelet additive solution (PAS), wherein the composition is free of platelets. In some embodiments, the concentration of the PIC is about 15 μM to about 1500 μM. In some embodiments, the PIC is a psoralen. In some embodiments, the PIC is amotosalen. In some embodiments, the PAS comprises one or more of chloride, acetate, citrate, potassium, magnesium, phosphate, gluconate, glucose, and bicarbonate. In some embodiments, the composition is sterile.

In another aspect, provided is a platelet composition prepared by any of the methods provided herein.

These and other aspects and advantages of the present disclosure will become apparent from the subsequent detailed description and the appended claims. It is to be understood that one, some, or all of the properties of the various embodiments described herein may be combined to form other embodiments of the present disclosure.

### BRIEF DESCRIPTION OF THE DRAWINGS

FIG. 1A shows an exemplary kit for preparing a platelet composition. Dotted lines indicate alternative points of addition for a preparation of platelets.

FIG. 1B shows an exemplary kit for preparing a platelet composition. Dotted lines indicate alternative points of addition for a preparation of platelets.

FIG. 1C shows an exemplary kit for preparing a platelet composition. Dotted lines indicate alternative points of addition for a preparation of platelets.

FIG. 1D shows an exemplary kit for preparing a platelet composition. Dotted lines indicate alternative points of addition for a preparation of platelets.

FIG. 1E shows an exemplary kit for preparing a platelet composition. Dotted line indicates a point of addition for a preparation of platelets.

FIG. 2A shows exemplary kits for preparing a platelet composition. Dotted lines indicate alternative points of addition for a preparation of platelets.

FIG. 2B shows an exemplary kit for preparing a platelet composition. Dotted lines indicate alternative points of addition for a preparation of platelets.

FIG. 2C shows an exemplary kit for preparing a platelet composition. Dotted lines indicate alternative points of addition for a preparation of platelets.

FIG. 2D shows an exemplary kit for preparing a platelet composition. Dotted lines indicate alternative points of addition for a preparation of platelets.

FIG. 2E shows an exemplary kit for preparing a platelet composition. Dotted line indicates a point of addition for a preparation of platelets.

FIG. 3 shows a container comprising a solution comprising a platelet additive solution (PAS) and a pathogen inactivation compound (PIC) connected to an exemplary apheresis device.

### DETAILED DESCRIPTION

The present disclosure provides, in some aspects, improved methods, kits, and compositions for pathogen inactivation of a preparation of platelets, including an apheresis-derived preparation of platelets, for preparing a platelet composition suitable for infusion.

The methods, kits, and compositions disclosed herein relate to dosing a pathogen inactivation compound (PIC), such as a photochemical compound, e.g., amotosalen, into a preparation of platelets at a fixed concentration of the PIC for pathogen inactivation. For example, the disclosure provides for pre-mixing the PIC with a platelet additive solution (PAS) at a desired (e.g., standardized) concentration and then dosing the PIC/PAS solution into a platelet preparation, thus allowing for, e.g., (i) improved processing flexibility and control, (ii) improved pathogen inactivation, including for example, allowing for reduced amounts of PIC used for pathogen inactivation, (iii) reduced processing steps, such as no requirement for further processing with a compound absorption device (CAD) to remove residual PIC or photoproducts thereof prior to administration to an individual, and/or (iv) improved platelet quality. Addition of a pre-mixed PIC/PAS solution in standard volumes that are multiples of a single, double and triple volume, i.e., 1×, 2× and 3×, may help streamline both the collection, e.g., via coupling with apheresis collection of platelets, and the treatment processes so that all therapeutic pathogen inactivated doses of platelets in, e.g., 65/35 PAS/plasma, are identical regardless whether they came from a single, double, or triple donation and can always be treated with the same concentration of PIC.

A number of benefits may be obtained through the improved methods, kits, and compositions disclosed herein, such as increasing standardization of treatment conditions that provide for pathogen inactivation with more consistent PIC concentrations, eliminating some of the restrictive guard bands for platelet volume and/or platelet concentration inputs, providing greater flexibility for treatment options available for pathogen inactivation of preparations of platelets, and/or reducing amounts of PIC needed for pathogen inactivation. The disclosure thus may allow for much more variation in donation volumes processed. This in turn may also provide for reduced variation in downstream processing steps (e.g., processing with a compound adsorption device (CAD)) and ultimately less variation in residual PIC in the final platelet product (e.g., platelet composition).

Additionally, utilizing a pre-mixed PIC/PAS may provide an opportunity to separately manufacture and/or supply the PIC component from the other components of disposable processing sets or as non-integrated components supplied with the processing sets (e.g., as kits), thereby greatly simplifying and reducing cost of goods for disposable sets associated with manufacturing processes. For example, the methods, kits, and compositions disclosed herein may provide for processing sets with separate/not connected “wet” side components (e.g., with PIC and PAS) and “dry” side components (e.g., illumination, CAD, and/or storage containers), thus simplifying manufacturing and sterilization risks thereof.

Moreover, the methods, kits, and compositions disclosed herein may allow for improved (e.g., increased) pathogen inactivation, for example, in variety of types or species of pathogens inactivated and/or the degree of pathogen inactivation of a single type or species of pathogen, and/or pathogen inactivation with reduced concentrations of PIC, e.g., via pre-incubation of PIC with a preparation of platelets.

### Definitions

“Preparation of platelets,” as used herein, means a composition comprising platelets that has not been subjected to a pathogen inactivation process. In some embodiments, a preparation of platelets is a platelet donation. In some embodiments, the preparation of platelets is obtained from an apheresis donation. In some embodiments, the preparation of platelets is obtained from a whole blood donation (e.g., by a buffy coat method, by a platelet rich plasma (PRP) method). In some embodiments, the preparation of platelets is obtained from more than one donor. In some embodiments, the preparation of platelets comprises plasma.

“Pathogen inactivation process,” as used herein, means a process useful for inactivating pathogens that may be present in a preparation of platelets, such as a platelet donation, where it is understood that the process does not necessarily inactivate completely all pathogens that may be present, but substantially reduces the amount of pathogens to significantly reduce the risk of a transfusion-associated disease. The inactivation of a pathogen may be assayed, for example, by measuring the number of infective pathogens (e.g., virus or bacteria) in a certain volume, and the level of inactivation is typically represented by the log reduction in the infectivity of the pathogen, or log reduction in titer. Methods of assaying log reduction in titer, and measurements thereof for pathogen inactivation are known in the art. Methods of assaying log reduction in titer and measurements thereof for pathogen inactivation are described, for example, in U.S. Pat. No. 7,655,392, the disclosure of which is hereby incorporated by reference as it relates to assays for pathogen inactivation. As such, for any given pathogen, known amounts can be added to a test unit of platelets (e.g., preparation of platelets) to assess how much inactivation results from the process, where typically the pathogen inactivation process results in at least about 1 log reduction in titer, or about 2 log, about 3 log, about 4 log, or at least about 5 log or greater reduction in titer. While the methods as described herein are applicable to any pathogen inactivation process, it is desirable that the pathogen inactivation process is capable of inactivating a variety of pathogens to at least 1 log reduction in titer, including a pathogen selected from the group consisting of HIV-1, HBV, HCV, HTLV-1, HTLV-2, West Nile virus, Hepatitis E virus, Escherichia coli, Klebsiella pneumoniae, Yersinia enterocolitica, Staphylococcus epidermidis, Staphylococcus aureus, Treponema pallidum, Borrelia burgdorferi, Plasmodium falciparum, Trypanosoma cruzi, and Babesia microti. In certain embodiments, a pathogen inactivation process may comprise treating with a pathogen inactivation compound (PIC).

“Pathogen inactivation compound” or “PIC,” as used herein, means any suitable compound, such as a small organic compound, that can be used to inactivate a pathogen and that may be present in a platelet-containing blood product. A “photoactivated pathogen inactivation compound” is a suitable compound that requires some level of light in order to sufficiently inactivate (e.g., photochemically inactivate) a pathogen. Such compounds are useful in the inactivation of pathogens in platelet or other blood products as they provide control over the inactivation process. Such photoactivated pathogen inactivation compounds described herein include psoralens, isoalloxazines, alloxazines, phthalocyanines, phenothiazines, and porphyrins, where these terms are understood to encompass a general class of compounds, i.e., the core compound and suitable derivatives thereof. For example psoralens or a psoralen generally describes the psoralen core compound and any derivative thereof (e.g., amotosalen), isoalloxazines, or an isoalloxazine generally describes the isoalloxazine core and any derivative thereof (e.g., riboflavin), and so forth. Such derivatives comprise the core compound structure as well as additional substituents on the core. Descriptions of such compounds include any salts thereof.

The term “amotosalen,” as used herein, means the compound 3-(2-aminoethoxymethyl)-2,5,9-trimethylfuro [3,2-g]chromen-7-one and any salts thereof. The amotosalen compound may also be referred to as 3-[(2-aminoethoxy)methyl]-2,5,9-trimethyl-7H-furo[3,2-G][1]benzopyran-7-one-hydrochloride. The amotosalen compound may also be referred to as 4′-(4-amino-2-oxa)butyl-4,5′,8-trimethyl psoralen. Where the inactivation of blood products such as a preparation of platelets includes adding amotosalen HCl (the HCl salt of amotosalen) to a blood product, the removal of this compound from the blood product is not limited to the removal of amotosalen HCl, as the amotosalen can be present in solution as other salts or as the free base.

“Platelet composition,” as used herein, means a pathogen-inactivated composition comprising platelets.

“Pathogen-inactivated” as used herein describes a blood product (e.g., a platelet composition) that has undergone a pathogen inactivation process (e.g., by the methods described herein) to inactivate pathogens that may be present. It is understood that the pathogen inactivation process does not necessarily inactivate completely all pathogens that may be present, but substantially reduces the amount of one or more pathogens to significantly reduce the risk of a transfusion-associated disease.

The term “suitable for infusion” refers to any blood product (e.g., platelet composition, pathogen inactivated platelet composition) able to be used for an infusion (e.g., a transfusion) into a subject (e.g., a human patient) according to medical judgement. In some embodiments, suitability refers to having sufficient biological activity for its intended use, i.e., for use where a transfusion of human coagulation factors is indicated, including, without limitation, control of bleeding associated with fibrinogen deficiency, treating Factor XIII deficiency, treating Factor VIII deficiency, treating von Willebrand disease, maintenance of hemostasis, treating disseminated intravascular coagulation (DIC) or high volume hemorrhage, and/or making fibrin sealant. In some embodiments, suitability refers to having sufficient safety, e.g., that the product has undergone a treatment that improves product safety (e.g., pathogen inactivation) and/or demonstrates satisfactory performance with respect to one or more safety-related measurements (such as viral or bacterial titer). Photochemical inactivation of pathogens in blood product units using amotosalen and UVA light as described herein is well established to provide such a blood product (e.g., platelet composition) that is suitable for infusion into humans. In some embodiments, suitability refers to meeting one or more standards (e.g., having a level of a biological activity or a biological component, a safety criterion, and the like) established by an accrediting agency or regulatory body that governs infusion practices, such as the AABB. In some embodiments, suitability of a platelet composition subjected to pathogen inactivation (e.g., photochemical pathogen inactivation, with amotosalen/UVA light) refers to a platelet composition with the concentration of PIC (e.g., residual PIC) below a certain level after the pathogen inactivation process.

The term “under sterile conditions” or “sterilely” as used herein refers to maintaining the sterility of the system, for example by connection of two bags from a blood processing set, or refers to a means by which the process does not introduce contamination. For example, as used in the methods described herein, a source unit of blood product such as a preparation of platelets (e.g., in a suitable container) comprising a tubing for connection to a processing set or container of pathogen inactivation compound comprising a similar tubing may be joined under sterile condition by methods known in the art, for example using a sterile connecting device, which acts to melt or weld the tubing together to provide a sterile flow path between the two containers. Similarly, when methods described herein describe sealing off such tubing, the sealing is done under sterile conditions, for example using a tubing welder.

### Methods of Preparing a Platelet Composition

The present disclosure provides, in some aspects, methods of preparing a platelet composition (e.g., pathogen inactivated platelet composition), comprising: (a) providing (e.g., in a first container) a solution comprising a platelet additive solution (PAS) and a pathogen inactivation compound (PIC); (b) admixing the solution of step (a) with a preparation of platelets; and (c) subjecting the admixture of step (b) to light sufficient to photochemically inactivate a pathogen, if present, thereby yielding the platelet composition.

The methods of preparing a platelet composition (e.g., pathogen inactivated platelet composition) disclosed herein, comprise (a) providing a solution comprising a platelet additive solution (PAS) and a pathogen inactivation compound (PIC), wherein the solution comprising the PAS and the PIC is of a sufficient volume for preparing any number of platelet compositions (e.g., platelet unit or therapeutic dose). In some embodiments, the first container of step (a) contains a sufficient volume of a solution comprising a platelet additive solution (PAS) and a pathogen inactivation compound (PIC) for preparing one platelet composition (e.g., platelet unit, therapeutic dose). In some embodiments, the first container of step (a) contains a sufficient volume of a solution comprising a platelet additive solution (PAS) and a pathogen inactivation compound (PIC) for preparing two or more (e.g., three) platelet compositions. In some embodiments, the first container of step (a) contains a sufficient volume of a solution comprising a platelet additive solution (PAS) and a pathogen inactivation compound (PIC) for preparing a platelet composition from one platelet donor. In some embodiments, the first container of step (a) contains a sufficient volume of a solution comprising a platelet additive solution (PAS) and a pathogen inactivation compound (PIC) for preparing platelet compositions from two or more platelet donors.

In some embodiments, provided is a method comprising: (a) providing in a first container a solution comprising a platelet additive solution (PAS) and a pathogen inactivation compound (PIC); (b) admixing in the first container the solution of step (a) with a preparation of platelets; and (c) subjecting the admixture of step (b) to light sufficient to photochemically inactivate a pathogen, if present, thereby yielding a platelet composition. In some embodiments, the first container is made of a material that is substantially translucent to light in the photochemical inactivation wavelength range (e.g., about 200 nm to about 400 nm, ultraviolet A spectrum), and the admixture of step (b) is subjected to the light in the first container. In some embodiments, the solution comprising a PAS and a PIC are combined with the preparation of platelets in the admixing of step (b) and incubated for a period of from 30 minutes to 24 hours before subjecting the admixture to light of step (c). In some embodiments, the first container comprises a compound adsorption device (CAD). In some embodiments, the first container is suitable for storing a platelet composition. In some embodiments, the method further comprises, following step (c), transferring (e.g., sterilely) the platelet composition to a container comprising a CAD. In some embodiments, the container comprising the CAD is suitable for storing the platelet composition. In some embodiments, the method further comprises, following step (c), transferring (e.g., sterilely) the platelet composition to at least one (e.g., 1, 2, or 3) container suitable for storing the platelet composition.

In some embodiments, provided is a method comprising: (a) providing in a first container a solution comprising a platelet additive solution (PAS) and a pathogen inactivation compound (PIC); (b) admixing in a second container the solution of step (a) with a preparation of platelets; and (c) subjecting the admixture of step (b) to light sufficient to photochemically inactivate a pathogen, if present, thereby yielding a platelet composition. In some embodiments, the second container is made of a material that is substantially translucent to light in the photochemical inactivation wavelength range (e.g., about 200 nm to about 400 nm, ultraviolet A spectrum), and the admixture of step (b) is subjected to the light in the second container. In some embodiments, the solution comprising a PAS and a PIC are combined with the preparation of platelets in the admixing step (b) and incubated for a period of from 30 minutes to 24 hours before subjecting the admixture to light of step (c). In some embodiments, the second container comprises a compound adsorption device (CAD). In some embodiments, the second container is suitable for storing a platelet composition. In some embodiments, the method further comprises, following step (c), transferring (e.g., sterilely) the platelet composition to a container comprising a CAD. In some embodiments, the container comprising the CAD is suitable for storing the platelet composition. In some embodiments, the method further comprises, following step (c), transferring (e.g., sterilely) the platelet composition to at least one (e.g., 1, 2, or 3) container suitable for storing the platelet composition.

In some embodiments, provided is a method comprising: (a) providing in a first container a solution comprising a platelet additive solution (PAS) and a pathogen inactivation compound (PIC); (b) connecting the first container to an apheresis device; (c) admixing in the first container the solution of step (a) with a preparation of platelets; and (d) subjecting the admixture of step (c) to light sufficient to photochemically inactivate a pathogen, if present, thereby yielding a platelet composition. In some embodiments, first container is sterilely connected to the apheresis device. In some embodiments, the first container is connected to a fluid flow path or channel of the apheresis device. In some embodiments, the first container is made of a material that is substantially translucent to light in the photochemical inactivation wavelength range (e.g., about 200 nm to about 400 nm, ultraviolet A spectrum), and the admixture of step (c) is subjected to the light in the first container. In some embodiments, the solution comprising a PAS and a PIC are combined with the preparation of platelets in the admixing of step (c) and incubated for a period of from 30 minutes to 24 hours before subjecting the admixture to light of step (d). In some embodiments, the first container comprises a compound adsorption device (CAD). In some embodiments, the first container is suitable for storing a platelet composition. In some embodiments, the method further comprises, following step (d), transferring (e.g., sterilely) the platelet composition to a container comprising a CAD. In some embodiments, the container comprising the CAD is suitable for storing the platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) providing in a first container a solution comprising a platelet additive solution (PAS) and a pathogen inactivation compound (PIC); (b) connecting the first container to an apheresis device; (c) admixing in a second container the solution of step (a) with a preparation of platelets; and (d) subjecting the admixture of step (c) to light sufficient to photochemically inactivate a pathogen, if present, thereby yielding a platelet composition. In some embodiments, the first container is sterilely connected to the apheresis device. In some embodiments, the first container is connected to a fluid flow path or channel of the apheresis device. In some embodiments, the second container is made of a material that is substantially translucent to light in the photochemical inactivation wavelength range (e.g., about 200 nm to about 400 nm, ultraviolet A spectrum), and the admixture of step (c) is subjected to the light in the second container. In some embodiments, the solution comprising a PAS and a PIC are combined with the preparation of platelets in the admixing of step (c) and incubated for a period of from 30 minutes to 24 hours before subjecting the admixture to light of step (d). In some embodiments, the second container comprises a compound adsorption device (CAD). In some embodiments, the second container is suitable for storing a platelet composition. In some embodiments, the method further comprises, following step (d), transferring (e.g., sterilely) the platelet composition to a container comprising a CAD. In some embodiments, the container comprising the CAD is suitable for storing the platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) providing in a first container a solution comprising a platelet additive solution (PAS) and a pathogen inactivation compound (PIC); (b) connecting the first container and a second container to an apheresis device; (c) admixing in the second container the solution of step (a) with a preparation of platelets; and (d) subjecting the admixture of step (c) to light sufficient to photochemically inactivate a pathogen, if present, thereby yielding a platelet composition. In some embodiments, the first and/or second container is sterilely connected to the apheresis device. In some embodiments, the first and/or second container is connected to a fluid flow path or channel of the apheresis device. In some embodiments, the second container is made of a material that is substantially translucent to light in the photochemical inactivation wavelength range (e.g., about 200 nm to about 400 nm, ultraviolet A spectrum), and the admixture of step (c) is subjected to the light in the second container. In some embodiments, the solution comprising a PAS and a PIC are combined with the preparation of platelets in the admixing of step (c) and incubated for a period of from 30 minutes to 24 hours before subjecting the admixture to light of step (d). In some embodiments, the second container comprises a compound adsorption device (CAD). In some embodiments, the second container is suitable for storing a platelet composition. In some embodiments, the method further comprises, following step (d), transferring (e.g., sterilely) the platelet composition to a container comprising a CAD. In some embodiments, the container comprising the CAD is suitable for storing the platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) admixing in the first container the admixture of step (a) with a preparation of platelets; and (c) subjecting the admixture of step (b) to light sufficient to photochemically inactivate a pathogen, if present, thereby yielding a platelet composition. In some embodiments, the first container is made of a material that is substantially translucent to light in the photochemical inactivation wavelength range (e.g., about 200 nm to about 400 nm, ultraviolet A spectrum), and the admixture of step (b) is subjected to the light in the first container. In some embodiments, the solution of PAS and the solution of PIC combined in step (a) are combined with the preparation of platelets in the admixing of step (b) and incubated for a period of from 30 minutes to 24 hours before subjecting the admixture to light of step (c). In some embodiments, the first container comprises a compound adsorption device (CAD). In some embodiments, the first container is suitable for storing a platelet composition. In some embodiments, the method further comprises, following step (c), transferring (e.g., sterilely) the platelet composition to a container comprising a CAD. In some embodiments, the container comprising the CAD is suitable for storing the platelet composition. In some embodiments, the method further comprises, following step (c), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) admixing in a second container the admixture of step (a) with a preparation of platelets; and (c) subjecting the admixture of step (b) to light sufficient to photochemically inactivate a pathogen, if present, thereby yielding a platelet composition. In some embodiments, the second container is made of a material that is substantially translucent to light in the photochemical inactivation wavelength range (e.g., about 200 nm to about 400 nm, ultraviolet A spectrum), and the admixture of step (b) is subjected to the light in the second container. In some embodiments, the solution of PAS and the solution of PIC combined in step (a) are combined with the preparation of platelets in the admixing of step (b) and incubated for a period of from 30 minutes to 24 hours before subjecting the admixture to light of step (c). In some embodiments, the second container comprises a compound adsorption device (CAD). In some embodiments, the second container is suitable for storing a platelet composition. In some embodiments, the method further comprises, following step (c), transferring (e.g., sterilely) the platelet composition to a container comprising a CAD. In some embodiments, the container comprising the CAD is suitable for storing the platelet composition. In some embodiments, the method further comprises, following step (c), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) connecting the first container to an apheresis device; (c) admixing in the first container the admixture of step (a) with a preparation of platelets; and (d) subjecting the admixture of step (c) to light sufficient to photochemically inactivate a pathogen, if present, thereby yielding a platelet composition. In some embodiments, the first container is sterilely connected to the apheresis device. In some embodiments, the first container is connected to a fluid flow path or channel of the apheresis device. In some embodiments, the first container is made of a material that is substantially translucent to light in the photochemical inactivation wavelength range (e.g., about 200 nm to about 400 nm, ultraviolet A spectrum), and the admixture of step (c) is subjected to the light in the first container. In some embodiments, the solution of PAS and the solution of PIC combined in step (a) are combined with the preparation of platelets in the admixing of step (c) and incubated for a period of from 30 minutes to 24 hours before subjecting the admixture to light of step (d). In some embodiments, the first container comprises a compound adsorption device (CAD). In some embodiments, the first container is suitable for storing a platelet composition. In some embodiments, the method further comprises, following step (d), transferring (e.g., sterilely) the platelet composition to a container comprising a CAD. In some embodiments, the container comprising the CAD is suitable for storing the platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) connecting the first container to an apheresis device; (c) admixing in a second container the admixture of step (a) with a preparation of platelets; and (d) subjecting the admixture of step (c) to light sufficient to photochemically inactivate a pathogen, if present, thereby yielding a platelet composition. In some embodiments, the first container is sterilely connected to the apheresis device. In some embodiments, the first container is connected to a fluid flow path or channel of the apheresis device. In some embodiments, the second container is made of a material that is substantially translucent to light in the photochemical inactivation wavelength range (e.g., about 200 nm to about 400 nm, ultraviolet A spectrum), and the admixture of step (c) is subjected to the light in the second container. In some embodiments, the solution of PAS and the solution of PIC combined in step (a) are combined with the preparation of platelets in the admixing of step (c) and incubated for a period of from 30 minutes to 24 hours before subjecting the admixture to light of step (d). In some embodiments, the second container comprises a compound adsorption device (CAD). In some embodiments, the second container is suitable for storing a platelet composition. In some embodiments, the method further comprises, following step (d), transferring (e.g., sterilely) the platelet composition to a container comprising a CAD. In some embodiments, the container comprising the CAD is suitable for storing the platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) connecting the first container and a second container to an apheresis device; (c) admixing in the second container the admixture of step (a) with a preparation of platelets; and (d) subjecting the admixture of step (c) to light sufficient to photochemically inactivate a pathogen, if present, thereby yielding a platelet composition. In some embodiments, the first and/or second container is sterilely connected to the apheresis device. In some embodiments, the first and/or second container is connected to a fluid flow path or channel of the apheresis device. In some embodiments, the second container is made of a material that is substantially translucent to light in the photochemical inactivation wavelength range (e.g., about 200 nm to about 400 nm, ultraviolet A spectrum), and the admixture of step (c) is subjected to the light in the second container. In some embodiments, the solution of PAS and the solution of PIC combined in step (a) are combined with the preparation of platelets in the admixing of step (c) and incubated for a period of from 30 minutes to 24 hours before subjecting the admixture to light of step (d). In some embodiments, the second container comprises a compound adsorption device (CAD). In some embodiments, the second container is suitable for storing a platelet composition. In some embodiments, the method further comprises, following step (d), transferring (e.g., sterilely) the platelet composition to a container comprising a CAD. In some embodiments, the container comprising the CAD is suitable for storing the platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) providing in a first container a solution comprising a platelet additive solution (PAS) and a pathogen inactivation compound (PIC); (b) admixing in the first container the solution of step (a) with a preparation of platelets; and (c) subjecting the admixture of step (b) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (c) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (c) comprises less than 5 μM of PIC (e.g., less than 2 μM of PIC). In some embodiments, the solution comprising a PAS and a PIC are combined with the preparation of platelets in the admixing of step (b) and incubated for a period of from 30 minutes to 24 hours before subjecting the admixture to light of step (c). In some embodiments, the first container is made of a material that is substantially translucent to light in the photochemical inactivation wavelength range (e.g., about 200 nm to about 400 nm, ultraviolet A spectrum), and the admixture of step (b) is subjected to the light in the first container. In some embodiments, the first container is suitable for storing a platelet composition. In some embodiments, the method further comprises, following step (c), transferring (e.g., sterilely) the platelet composition to at least one (e.g., 1, 2, or 3) container suitable for storing the platelet composition.

In some embodiments, provided is a method comprising: (a) providing in a first container a solution comprising a platelet additive solution (PAS) and a pathogen inactivation compound (PIC); (b) admixing in a second container the solution of step (a) with a preparation of platelets; and (c) subjecting the admixture of step (b) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (c) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (c) comprises less than 5 μM of PIC (e.g., less than 2 μM of PIC). In some embodiments, the solution comprising a PAS and a PIC are combined with the preparation of platelets in the admixing of step (b) and incubated for a period of from 30 minutes to 24 hours before subjecting the admixture to light of step (c). In some embodiments, the second container is made of a material that is substantially translucent to light in the photochemical inactivation wavelength range (e.g., about 200 nm to about 400 nm, ultraviolet A spectrum), and the admixture of step (b) is subjected to the light in the second container. In some embodiments, the second container is suitable for storing a platelet composition. In some embodiments, the method further comprises, following step (c), transferring (e.g., sterilely) the platelet composition to at least one (e.g., 1, 2, or 3) container suitable for storing the platelet composition.

In some embodiments, provided is a method comprising: (a) providing in a first container a solution comprising a platelet additive solution (PAS) and a pathogen inactivation compound (PIC); (b) connecting the first container to an apheresis device; (c) admixing in the first container the solution of step (a) with a preparation of platelets; and (d) subjecting the admixture of step (c) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (d) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (d) comprises less than 5 μM of PIC (e.g., less than 2 M of PIC). In some embodiments, the solution comprising a PAS and a PIC are combined with the preparation of platelets in the admixing of step (c) and incubated for a period of from 30 minutes to 24 hours before subjecting the admixture to light of step (d). In some embodiments, first container is sterilely connected to the apheresis device. In some embodiments, the first container is connected to a fluid flow path or channel of the apheresis device. In some embodiments, the first container is made of a material that is substantially translucent to light in the photochemical inactivation wavelength range (e.g., about 200 nm to about 400 nm, ultraviolet A spectrum), and the admixture of step (c) is subjected to the light in the first container. In some embodiments, the first container is suitable for storing a platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) providing in a first container a solution comprising a platelet additive solution (PAS) and a pathogen inactivation compound (PIC); (b) connecting the first container to an apheresis device; (c) admixing in a second container the solution of step (a) with a preparation of platelets; and (d) subjecting the admixture of step (c) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (d) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (d) comprises less than 5 μM of PIC (e.g., less than 2 M of PIC). In some embodiments, the solution comprising a PAS and a PIC are combined with the preparation of platelets in the admixing of step (c) and incubated for a period of from 30 minutes to 24 hours before subjecting the admixture to light of step (d). In some embodiments, the first container is sterilely connected to the apheresis device. In some embodiments, the first container is connected to a fluid flow path or channel of the apheresis device. In some embodiments, the second container is made of a material that is substantially translucent to light in the photochemical inactivation wavelength range (e.g., about 200 nm to about 400 nm, ultraviolet A spectrum), and the admixture of step (c) is subjected to the light in the second container. In some embodiments, the second container is suitable for storing a platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) providing in a first container a solution comprising a platelet additive solution (PAS) and a pathogen inactivation compound (PIC); (b) connecting the first container and a second container to an apheresis device; (c) admixing in the second container the solution of step (a) with a preparation of platelets; and (d) subjecting the admixture of step (c) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (d) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (d) comprises less than 5 μM of PIC (e.g., less than 2 μM of PIC). In some embodiments, the solution comprising a PAS and a PIC are combined with the preparation of platelets in the admixing of step (c) and incubated for a period of from 30 minutes to 24 hours before subjecting the admixture to light of step (d). In some embodiments, the first and/or second container is sterilely connected to the apheresis device. In some embodiments, the first and/or second container is connected to a fluid flow path or channel of the apheresis device. In some embodiments, the second container is made of a material that is substantially translucent to light in the photochemical inactivation wavelength range (e.g., about 200 nm to about 400 nm, ultraviolet A spectrum), and the admixture of step (c) is subjected to the light in the second container. In some embodiments, the second container is suitable for storing a platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) admixing in the first container the admixture of step (a) with a preparation of platelets; and (c) subjecting the admixture of step (b) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (c) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (c) comprises less than 5 μM of PIC (e.g., less than 2 μM of PIC). In some embodiments, the solution of PAS and the solution of PIC combined in step (a) are combined with the preparation of platelets in the admixing of step (b) and incubated for a period of from 30 minutes to 24 hours before subjecting the admixture to light of step (c). In some embodiments, the first container is made of a material that is substantially translucent to light in the photochemical inactivation wavelength range (e.g., about 200 nm to about 400 nm, ultraviolet A spectrum), and the admixture of step (b) is subjected to the light in the first container. In some embodiments, the first container is suitable for storing a platelet composition. In some embodiments, the method further comprises, following step (c), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) admixing in a second container the admixture of step (a) with a preparation of platelets; and (c) subjecting the admixture of step (b) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (c) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (c) comprises less than 5 μM of PIC (e.g., less than 2 μM of PIC). In some embodiments, the solution of PAS and the solution of PIC combined in step (a) are combined with the preparation of platelets in the admixing of step (b) and incubated for a period of from 30 minutes to 24 hours before subjecting the admixture to light of step (c). In some embodiments, the second container is made of a material that is substantially translucent to light in the photochemical inactivation wavelength range (e.g., about 200 nm to about 400 nm, ultraviolet A spectrum), and the admixture of step (b) is subjected to the light in the second container. In some embodiments, the second container is suitable for storing a platelet composition. In some embodiments, the method further comprises, following step (c), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) connecting the first container to an apheresis device; (c) admixing in the first container the admixture of step (a) with a preparation of platelets; and (d) subjecting the admixture of step (c) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (d) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (d) comprises less than 5 μM of PIC (e.g., less than 2 M of PIC). In some embodiments, the solution of PAS and the solution of PIC combined in step (a) are combined with the preparation of platelets in the admixing of step (c) and incubated for a period of from 30 minutes to 24 hours before subjecting the admixture to light of step (d). In some embodiments, the first container is sterilely connected to the apheresis device. In some embodiments, the first container is connected to a fluid flow path or channel of the apheresis device. In some embodiments, the first container is made of a material that is substantially translucent to light in the photochemical inactivation wavelength range (e.g., about 200 nm to about 400 nm, ultraviolet A spectrum), and the admixture of step (c) is subjected to the light in the first container. In some embodiments, the first container is suitable for storing a platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) connecting the first container to an apheresis device; (c) admixing in a second container the admixture of step (a) with a preparation of platelets; and (d) subjecting the admixture of step (c) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (d) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (d) comprises less than 5 μM of PIC (e.g., less than 2 M of PIC). In some embodiments, the solution of PAS and the solution of PIC combined in step (a) are combined with the preparation of platelets in the admixing of step (b) and incubated for a period of from 30 minutes to 24 hours before subjecting the admixture to light of step (d). In some embodiments, the first container is sterilely connected to the apheresis device. In some embodiments, the first container is connected to a fluid flow path or channel of the apheresis device. In some embodiments, the second container is made of a material that is substantially translucent to light in the photochemical inactivation wavelength range (e.g., about 200 nm to about 400 nm, ultraviolet A spectrum), and the admixture of step (c) is subjected to the light in the second container. In some embodiments, the second container is suitable for storing a platelet composition. In some embodiments, the method further comprises, following step (d), transferring (e.g., sterilely) the platelet composition to at least one (e.g., 1, 2, or 3) container suitable for storing the platelet composition.

In some embodiments, provided is a method comprising: (a) combining (e.g., admixing) in a first container a platelet additive solution (PAS) and a pathogen inactivation compound (PIC); (b) connecting the first container and a second container to an apheresis device; (c) admixing in the second container the admixture of step (a) with a preparation of platelets; and (d) subjecting the admixture of step (c) to light sufficient to photochemically inactivate a pathogen, if present, thereby yielding a platelet composition, wherein the method is sufficient to inactivate at least 1 log of the pathogen (e.g., at least 4 logs of the pathogen), and wherein the platelet composition after step (d) is suitable for infusion into a subject without further processing, including without exposure to a compound adsorption device (CAD), to remove residual PIC or photoproducts thereof. In some embodiments, the method is sufficient to inactivate at least 1 log of a pathogen (e.g., at least 4 logs of a pathogen), and wherein the platelet composition after step (d) comprises less than 5 μM of PIC (e.g., less than 2 μM of PIC). In some embodiments, the solution of PAS and the solution of PIC combined in step (a) are combined with the preparation of platelets in the admixing of step (c) and incubated for a period of from 30 minutes to 24 hours before subjecting the admixture to light of step (d). In some embodiments, the first and/or second container is sterilely connected to the apheresis device. In some embodiments, the first and/or second container is connected to a fluid flow path or channel of the apheresis device. In some embodiments, the second container is made of a material that is substantially translucent to light in the photochemical inactivation wavelength range (e.g., about 200 nm to about 400 nm, ultraviolet A spectrum), and the admixture of step (c) is subjected to the light in the second container. In some embodiments, the second container is suitable for storing a platelet composition. In some embodiments, the method further comprises, following step (d), transferring (e.g., sterilely) the platelet composition to at least one (e.g., 1, 2, or 3) container suitable for storing the platelet composition.

In any or all of the aforementioned embodiments, providing in a first container a solution comprising a PAS and a PIC comprises first combining a solution of PAS and a solution of PIC to yield the solution comprising a PAS and a PIC. In any or all of the aforementioned embodiments, the method comprises, prior to step (a), combining a solution of PAS and a solution of PIC to yield a solution comprising a PAS and a PIC. In some embodiments, the solution of PAS is from a PAS container (e.g., PAS storage container). In some embodiments, the solution of PIC is from a PIC container (e.g., PIC storage container). In some embodiments, the solution of PAS and solution of PIC are combined in the first container of step (a). In some embodiments, the first container of step (a) is the PAS container. In some embodiments, the solution of PAS and the solution of PIC are combined less than 24 hours (e.g., within 24 hours) before the admixing of step (b). In some embodiments, the first container of step (a) is the PIC container. In some embodiments, the PAS container is connected to an apheresis device. In some embodiments, the PIC container is connected to an apheresis device.

In any or all of the aforementioned embodiments a container containing an admixture of platelet additive solution (PAS), pathogen inactivation compound (PIC) and preparation of platelets may be disconnected (e.g., sterilely disconnected) from an apheresis device prior to subjecting the admixture to light sufficient to photochemically inactivate a pathogen, if present.

The present disclosure provides, in some aspects, methods of preparing a platelet composition suitable for infusion into an individual from a preparation of platelets. In some embodiments of any of the methods, kits, and compositions described herein, one or more preparations of platelets are treated with the methods disclosed herein, thereby yielding one or more platelet compositions. In some embodiments, the method is sufficient to inactivate at least 1 log of a pathogen, and wherein the platelet composition after step (c) is suitable for infusion into a subject without further processing to remove residual PIC or photoproducts thereof. In some embodiments, the method is sufficient to inactivate at least 1 log of a pathogen, and wherein the platelet composition after step (c) comprises 5 μM or less of PIC. In some embodiments, the concentration of PIC in the admixture of step (b) is at least 10 μM.

### Preparations of Platelets

In some embodiments, the preparation of platelets is prepared from one or more, such as at least 1, at least 2, at least 3, at least 4, at least 5, at least 6, at least 7, at least 8, at least 9, or at least 10, apheresis-derived platelet donations. In some embodiments, the preparation of platelets is prepared from one or more, such as 1, 2, 3, 4, 5, 6, 7, 8, 9, or 10, apheresis-derived platelet donations. In some embodiments, the preparation of platelets is prepared from one or more, such as at least 1, at least 2, at least 3, at least 4, at least 5, at least 6, at least 7, at least 8, at least 9, or at least 10, whole blood-derived (e.g., PRP, buffy coat) platelet donations. In some embodiments, the preparation of platelets is prepared from one or more, such as 1, 2, 3, 4, 5, 6, 7, 8, 9, or 10, whole blood-derived (e.g., PRP, buffy coat) platelet donations.

### Apheresis Collected Platelets

In some embodiments of any of the methods, kits, and compositions described herein, the preparation of platelets is prepared by an apheresis method.

Apheresis methods generally refer to methods using an automated blood collection device (e.g., apheresis device) that uses centrifugal or filtration separation to automatically withdraw whole blood from a donor, separate the whole blood into blood components, collect certain of the components (e.g., platelets), and return to the donor some or all of the remainder of the whole blood and/or remaining uncollected blood components. Plateletpheresis is the collection of platelets using such an automated blood cell separator device, which results in obtaining a high yield of platelets (e.g., apheresis platelets) from a single donor. In some embodiments, a desired amount of plasma is maintained with the collected platelets. Some apheresis devices are capable of collection procedures not only for single platelet units, but also double and triple platelet units. Apheresis device may also include a container of anticoagulant from which the anticoagulant is metered into the flow path and mixed with the incoming whole blood. Anticoagulant is required because of the tendency of blood to clot and adhere to the walls of the plastic surfaces to which it comes in contact. Exemplary anticoagulants are well known in the art and may include, but are not limited to, an anticoagulant citrate phosphate dextrose (CPD) solution, an anticoagulant citrate phosphate double dextrose (CP2D) solution, an anticoagulant citrate phosphate dextrose adenine (CPDA) solution (e.g., CPDA-1), an acid citrate dextrose (ACD) solution (e.g., ACD-A), and an anticoagulant sodium citrate 4% w/v solution. Apheresis collection devices are well known in the art, with several such devices commercially available, including for example, the Amicus© system (Fenwal, Inc.), the Trima Accel® system (Terumo BCT) and the MCS®+ 9000 mobile system (Haemonetics, Inc.).

Apheresis platelet donations are based on certain donor parameters, such as for example, gender, physical size (e.g., weight), hemoglobin level, platelet count on the day of donation, prior donation history and donation frequency, in part to ensure only a safe amount of platelets is collected. Any or all of these parameters may be entered into a computer system and/or the apheresis collection device. From these parameters, apheresis platelet donations generally are collected from an individual donor as a volume to yield one, two or three platelet units (e.g., therapeutic dosage units) each containing a specified minimum number (e.g., at least a specified minimum number) of platelets per unit to meet the therapeutic dose requirement, with such per unit or therapeutic dose criteria generally determined by governmental, regulatory or accrediting organization (e.g., industry) standards. Non-limiting examples of such standards include, for example, those set forth by FDA, EDQM, AABB, PMDA, TGA and SFDA. The specified minimum, for example, may vary by country. Generally, a platelet number may be determined for each unit of a preparation of platelets, for example, based on a pre-donation platelet count and information about the volume collected, or alternatively by post-collection testing of units. In some embodiments, each unit of a preparation of platelets will comprise a minimum platelet number; however, determination of the platelet number for each unit may not be an absolute requirement and some platelet units in a plurality of platelet units may have less than a specified number.

### Whole Blood Collection and Processing of Platelets

In some embodiments of any of the methods, kits, and compositions described herein, the preparation of platelets is prepared from one or more whole blood donation(s) by a buffy coat method or a platelet rich plasma (PRP) method.

In some embodiments, the preparation of platelets is prepared from one or more whole blood donation(s) by a buffy coat method.

In some embodiments, the preparation of platelets is prepared from one or more whole blood donation(s) by a platelet rich plasma (PRP) method.

Whole blood for use in the preparation of platelets as described herein may be collected by a variety of procedures known in the art. One of the most common blood collection techniques is the “manual” collection of whole blood from donors. As commonly understood and as used herein, manual collection refers to a collection method where whole blood is allowed to drain from the donor and into a collection container without the use of external pumps or similar devices. This is in contrast to so-called automated procedures where blood is withdrawn from a donor and further processed by an instrument that typically includes a processing or separation device and pumps for moving blood or blood components into and out of the device.

Withdrawing blood from the donor typically includes inserting a vein access device, such as a needle, into the donor's arm (and, more specifically, the donor's vein) and withdrawing blood from the donor through the needle. The “venipuncture” needle typically has attached to it one end of a plastic tube that provides a flow path for the blood. The other end of the plastic tube terminates in one or more pre-attached plastic blood containers or bags for collecting the blood. The needle, tubing, and containers make up a blood collection set, which is pre-sterilized and disposed of after a single use. The sterile blood collection container typically serves as the primary container for initial separation of blood components (e.g., separation of plasma from red blood cells and platelets).

The blood collection container and plastic tubing may also include a volume of a liquid anticoagulant, while in the automated technique, a separate container of anticoagulant may be provided from which the anticoagulant is metered into the flow path and mixed with the incoming whole blood. Anticoagulant is required because of the tendency of blood to clot and adhere to the walls of the plastic surfaces which it. Exemplary anticoagulants are well known in the art and may include, but are not limited to, an anticoagulant citrate phosphate dextrose (CPD) solution, an anticoagulant citrate phosphate double dextrose (CP2D) solution, an anticoagulant citrate phosphate dextrose adenine (CPDA) solution (e.g., CPDA-1), an acid citrate dextrose (ACD) solution (e.g., ACD-A), and an anticoagulant sodium citrate 4% w/v solution.

Blood may be identified or characterized with respect to one or more parameters, such as for example, hematocrit. Such identification or characterization is typically prior to or shortly after blood collection, but prior to subjecting the collected whole blood to further processing, such as according to the methods provided herein. In addition, at or near the time of collection and prior to transfusion to a patient, tests may be performed for determining blood type and the presence of pathogens such as virus, bacteria and/or other foreign substances in the donor's blood. Such testing generally requires obtaining a sample of the donor's blood. Generally sampling of blood may be before, during or after donation, but without compromising the sterility of the system and/or the collected blood product. For example, samples may be commonly obtained by finger stick, heel stick, or venipuncture. In the case where blood for hemoglobin testing is gathered with a capillary stick, a single-use sterile lancet may be used. Another well-known technique is to simply withdraw or collect the blood remaining in the flow path of the collection set after donation. This involves removing the needle from the donor, inserting the needle into a vacuum sealed sampling vial or tube, and allowing the blood from the flow path to drain into the vial. Another alternative is to clamp off the flow path near the collection container and divert the blood being withdrawn from the donor to a collection (sampling) vial or tube. This procedure may employ a particular type of disposable tubing set having a pre-attached sampling site on the main flow path. Blood at or near the sampling site may be obtained by piercing the sampling site with a separately provided needle or other piercing device and attaching a sampling vial thereto. To minimize the risk that the incoming blood will be exposed to the outside environment, the sample is typically collected after completion of the blood donation. Alternatively, some collection bags or collection sets include diversion pouches to sequester a portion (e.g., the first 20 ml) of blood collected. Another example of a blood sampling system is described in U.S. Pat. No. 5,167,656, which is hereby incorporated by reference in its entirety, which describes blood collection sets with an enlarged sample collection portion included in the flow path. Blood for sampling is collected in the enlarged portion by clamping off the flow path near the collection container and allowing the enlarged tubing portion to fill with blood.

Buffy coat methods are known in the art. Buffy coat methods comprise separating blood components of uncoagulated blood samples via centrifugation to obtaining a layer comprising plasma, a layer comprising erythrocytes, and a layer (i.e., buffy coat) comprising platelets and leukocytes. Following centrifugation, the buffy coat may be isolated from the other blood components to obtain a preparation of platelets.

Platelet rich plasma (PRP) methods are known in the art. PRP methods comprise separating blood components of uncoagulated blood samples via centrifugation to obtain a layer comprising erythrocytes and a layer comprising plasma and platelets. Following centrifugation, the layer of plasma and platelets may be isolated from the other blood components (and optionally further centrifuged to concentrate the platelets) to obtain a preparation of platelets.

### Pathogen Inactivation Compound (PIC)

In some embodiments of any of the methods, kits, and compositions provided herein, pathogen inactivation requires addition of an amount of pathogen inactivation compound (e.g., to a preparation of platelets). For example, pathogen inactivation may involve the addition of a low molecular weight compound that inactivates various pathogens, where a particular method involves the addition of a photosensitizer that, when activated by illumination using light of suitable wavelengths, will inactivate a variety of pathogens that may be present. Two methods that are commercially available include the addition of amotosalen or riboflavin to the platelets, with subsequent illumination with UV light. Other methods include illumination with other photoactive compounds, including psoralen derivatives other than amotosalen, isoalloxazines other than riboflavin, alloxazines, dyes such as phthalocyanines, phenothiazine dyes (e.g. methylene blue, azure B, azure C, thionine, toluidine blue), porphyrin derivatives (e.g. dihematoporphyrin ether, hematoporphyrin derivatives, benzoporphyrin derivatives, alkyl-substituted sapphyrin), and merocyanine 540 (Prodouz et al., Blood Cells 1992, 18(1):101-14; Sofer, Gail, BioPharm, August 2002). In some embodiments, the pathogen inactivation compound is a photoactive pathogen inactivation. In some embodiments, the pathogen inactivation compound (PIC) is a psoralen. In some embodiments, the pathogen inactivation compound (PIC) is amotosalen. In some embodiments, the pathogen inactivation compound (PIC) is selected from the group consisting of an isoalloxazine, an alloxazine, a phthalocyanine, a phenothiazine, a porphyrin, merocyanine 540, and salts or free bases thereof.

### Platelet Additive Solution (PAS)

Platelet additive solutions are known in the art, for example, as described by Alhumaidan et al. and Ringwald et al. (Alhumaidan, H. and Sweeney, J., J Clin Apheresis, 27: 93-98 (2012); Ringwald et al., Transfusion Medicine Reviews, 20: 158-64 (2006)), which are hereby incorporated by reference in their entirety. In some embodiments of any of the methods, kits, and compositions provided herein, the platelet additive solution (PAS) comprises one or more of chloride, acetate, citrate, potassium, magnesium, phosphate, gluconate, glucose, and bicarbonate. In some embodiments of any of the methods, kits, and compositions provided herein, the platelet additive solution (PAS) is a PAS approved by a regulatory agency or accrediting organization generally accepted in the field.

In some embodiments of any of the methods, kits, and compositions provided herein, the platelet additive solution (PAS) comprises one or more of sodium chloride, sodium acetate, sodium citrate, potassium chloride, magnesium chloride, sodium phosphate, sodium gluconate, glucose, and sodium bicarbonate.

In some embodiments, the PAS comprises chloride, citrate, phosphate, and potassium. In some embodiments, the PAS comprises chloride, citrate, and acetate. In some embodiments, the PAS comprises chloride, citrate, phosphate, and acetate. In some embodiments, the PAS comprises chloride, citrate, acetate, magnesium, potassium, and gluconate. In some embodiments, the PAS comprises chloride, citrate, phosphate, acetate, magnesium, and potassium. In some embodiments, the PAS comprises chloride, acetate, magnesium, potassium, and gluconate. In some embodiments, the PAS comprises chloride, citrate, phosphate, acetate, magnesium, potassium, and glucose.

In some embodiments, the PAS comprises sodium chloride, sodium acetate, potassium chloride, magnesium chloride, and sodium gluconate. In some embodiments, the PAS comprises sodium chloride, sodium acetate, and sodium citrate. In some embodiments, the PAS comprises sodium chloride, sodium acetate, sodium citrate, and sodium phosphate. In some embodiments, the PAS comprises sodium chloride, sodium citrate, sodium phosphate, and potassium chloride. In some embodiments, the PAS comprises sodium chloride, sodium acetate, sodium citrate, potassium chloride, magnesium chloride, and sodium phosphate. In some embodiments, the PAS comprises sodium chloride, sodium acetate, sodium citrate, potassium chloride, magnesium chloride, and sodium gluconate. In some embodiments, the PAS comprises sodium chloride, sodium acetate, sodium citrate, potassium chloride, magnesium chloride, sodium phosphate, glucose, and sodium bicarbonate. In some embodiments, the PAS comprises sodium chloride, sodium acetate, sodium citrate, potassium chloride, magnesium chloride, glucose, and sodium bicarbonate.

In some embodiments, the PAS is PAS-I. In some embodiments, the PAS is PlasmaLyte. In some embodiments, the PAS is Pas-II. In some embodiments, the PAS is T-Sol. In some embodiments, the PAS is PAS-III. In some embodiments, the PAS is Intersol. In some embodiments, the PAS is PAS-IIIM SSP. In some embodiments, the PAS is ComposolPAS-G. In some embodiments, the PAS is M-Sol. In some embodiments, the PAS is Isoplate. In some embodiments, the PAS is PAS-A. In some embodiments, the PAS is PAS-B. In some embodiments, the PAS is PAS-C. In some embodiments, the PAS is PAS-D. In some embodiments, the PAS is PAS-E. In some embodiments, the PAS is PAS-F. In some embodiments, the PAS is PAS-G.

### Solution of PAS and PIC

Generally, the solution comprising a PAS and a PIC can be of any volume sufficient for use in any of the methods, kits, and compositions described herein. In some embodiments of any of the methods, kits, and compositions described herein, the solution comprising a PAS and a PIC has a volume of between about 100 mL and about 1000 mL. In some embodiments, the solution comprising a PAS and a PIC has a volume of between about 200 mL and about 900 mL, between about 300 mL and about 800 mL, between about 400 mL and about 700 mL, or between about 500 mL and about 600 mL. In some embodiments, the solution comprising a PAS and a PIC has a volume of about 100 mL, about 200 mL, about 300 mL, about 400 mL, about 500 mL, about 600 mL, about 700 mL, about 800 mL, about 900 mL, or about 1000 mL. In some embodiments, the solution comprising a PAS and a PIC has a volume of less than about 1000 mL, less than about 800 mL, less than about 600 mL, less than about 500 mL, less than about 400 mL, less than about 300 mL, or less than about 200 mL. In some embodiments, the solution comprising a PAS and a PIC has a volume of greater than about 800 mL, greater than about 700 mL, greater than about 600 mL, greater than about 500 mL, greater than about 400 mL, greater than about 300 mL, greater than about 200 mL, or greater than about 100 mL. In some embodiments, the solution comprising a PAS and a PIC has a volume of between about 1000 mL and about 5000 mL.

Generally, the concentration of PIC in the solution comprising a PAS and a PIC can be any suitable concentration of PIC that provides for a “final” desired concentration of PIC upon mixing the solution comprising PAS and PIC with a preparation of platelets, for use in any of the methods, kits, and compositions described herein, such as for example taking into account the volumes to be combined when mixing the solution comprising PAS and PIC and the preparation of platelets. In some embodiments, the concentration of PIC in the solution comprising a PAS and a PIC is about 25 μM to about 1200 μM, about 50 μM to about 1000 μM, about 50 μM to about 750 μM, about 50 μM to about 500 μM, about 75 μM to about 500 μM, about 100 μM to about 400 PM, about 150 μM to about 350 μM, about 200 μM to about 300 μM, or about 225 μM to about 250 μM. In some embodiments, the concentration of PIC in the solution comprising a PAS and a PIC is about 25 μM, about 50 μM, about 75 μM, about 100 μM, about 125 μM, about 150 PM, about 175 μM, about 200 μM, about 250 μM about 275 μM, about 300 μM, about 325 PM, about 350 PM, about 375 μM, about 400 μM, about 450 μM, about 500 μM, about 550 PM, about 600 PM, about 650 μM, about 700 μM, about 750 μM, about 800 μM, about 850 μM, about 900 μM, about 1000 μM, about 1100 μM, about 1200 μM, about 1300 μM, about 1400 μM, or about 1500 μM. In some embodiments, the concentration of PIC in the solution comprising a PAS and a PIC is about 225 μM to about 235 μM. In some embodiments, the concentration of PIC in the solution comprising a PAS and a PIC is about 225 μM, about 226 μM, about 227 μM, about 228 μM, about 229 PM, about 230 μM, about 231 μM, about 232 μM, about 233 μM, about 234 μM, or about 235 μM.

In some embodiments, the solution comprising a PAS and a PIC is from combining a solution of PAS and a solution of PIC to yield the solution comprising a PAS and a PIC. In some embodiments, the method comprises, prior to step (a), combining a solution of PAS and a solution of PIC to yield a solution comprising a PAS and a PIC. In some embodiments, the solution of PAS is from a PAS container (e.g., PAS storage container). In some embodiments, the solution of PIC is from a PIC container (e.g., PIC storage container). In some embodiments, the solution of PAS and the solution of PIC are combined less than (e.g., within) about 6 months, less than about 4 months, less than about 3 months, less than about 2 months, less than about 1 month, less than about 3 weeks, less than about 2 weeks, less than about 1 week, less than about 5 days, less than about 4 days, less than about 3 days, less than about 48 hours, less than about 36 hours, less than about 24 hours, less than about 18 hours, less than about 12 hours, less than about 8 hours, less than about 6 hours, less than about 4 hours, less than about 2 hours, or less than about 1 hour before admixing the solution comprising a PAS and a PIC with a preparation of platelets. In some embodiments, the solution of PAS and the solution of PIC are combined about 5 minutes to about 72 hours, about 5 minutes to about 48 hours, about 5 minutes to about 36 hours, about 5 minutes to about 24 hours, about 5 minutes to about 18 hours, about 5 minutes to about 12 hours, about 5 minutes to about 8 hours, about 5 minutes to about 6 hours, about 5 minutes to about 4 hours, about 5 minutes to about 2 hours, or about 5 minutes to about 1 hour before admixing the solution comprising a PAS and a PIC with a preparation of platelets.

### Admixture of PAS, PIC, and Preparation of Platelets

In some embodiments of any of the methods, kits, and compositions described herein, the volume of an admixture of a PAS, a PIC, and a preparation of platelets is about 100 mL to about 1000 mL. In some embodiments, the volume of an admixture of a PAS, a PIC, and a preparation of platelets is about 200 mL to about 800 mL, about 200 mL to about 775 mL, about 250 mL to about 775 mL, about 225 mL to about 525 mL, about 500 mL to about 775 mL, about 200 mL to about 300 mL, about 300 mL to about 400 mL, about 400 mL to about 500 mL, about 500 mL to about 600 mL, about 600 mL to about 700 mL, or about 700 mL to about 800 mL. In some embodiments, the volume of an admixture of a PAS, a PIC, and a preparation of platelets is about 255 mL, 510 mL, or about 765 mL. In some embodiments, the admixture of a PAS, a PIC, and a preparation of platelets, wherein the preparation of platelets comprises plasma, has a ratio of the PAS to plasma of about 65:35.

In some embodiments, the total volume of an admixture of a PAS, a PIC, and a preparation of platelets, wherein the preparation of platelets comprises plasma, comprises about 32% to about 47% by volume plasma. In some embodiments, the total volume of an admixture of a PAS, a PIC, and a preparation of platelets, wherein the preparation of platelets comprises plasma, comprises about 53% to about 63% by volume PAS. In some embodiments, the plasma comprises about 32 to 47% by volume of an admixture of a PAS, a PIC, and a preparation of platelets, with platelet additive solution (e.g., platelet additive solution with PIC) comprising the remaining volume (i.e., 53 to 68% PAS, where % plasma+% PAS=100). The plasma volume may also include, for example, any volume that is not PAS (e.g., PAS with PIC), such as for example any volume associated with the platelets and/or any volume associated with an anticoagulant used during processing. In some embodiments, the preparation of platelets comprises plasma of about 32%, about 33%, about 34%, about 35%, about 36%, about 37%, about 38%, about 39%, about 40%, about 41%, about 42%, about 43%, about 44%, about 45%, about 46%, or about 47% by volume of the admixture of a PAS, a PIC, and a preparation of platelets. In some embodiments, the ratio of PAS to plasma by volume in the admixture of a PAS, a PIC, and a preparation of platelets is about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, or about 53:47.

### Pathogen Inactivation

In some embodiments of any of the methods, kits, and compositions described herein, the admixture of a PAS, a PIC, and a preparation of platelets comprises the PIC at a concentration sufficient to result in inactivation of at least about 1 log of a pathogen, if present. In some embodiments, the admixture of a PAS, a PIC, and a preparation of platelets comprises the PIC at a concentration sufficient to result in inactivation of at least about 1 log of a pathogen, if present, after the admixture is exposed to light sufficient to photochemically inactivate the pathogen. In some embodiments, the concentration of PIC is sufficient to result in inactivation of at least about 1 log, at least about 2 logs, at least about 3 logs, at least about 4 logs, at least about 5 logs, at least about 6 logs, or at least about 7 logs, at least about 8 logs, at least about 9 logs, or at least about 10 logs, of a pathogen, if present (e.g., after the admixture is exposed to light sufficient to photochemically inactivate the pathogen).

In some embodiments of any of the methods, kits, and compositions described herein, the admixture of a PAS, a PIC, and a preparation of platelets comprises the PIC at a concentration of about 5 μM to about 500 μM. In some embodiments, the admixture comprises the PIC at a concentration of less than about 150 μM. In some embodiments, the admixture comprises the PIC at a concentration of about 15 μM to about 400 μM, about 25 μM to about 300 PM, about 50 μM to about 250 μM, about 75 μM to about 225 μM, about 100 μM to about 200 PM, about 125 μM to about 175 μM, about 25 μM to about 250 μM, about 25 μM to about 200 PM, about 25 μM to about 150 μM, about 25 μM to about 100 μM, about 25 μM to about 50 μM, about 25 μM to about 35 μM, about 30 μM to about 150 μM, about 30 μM to about 90 μM, about 50 μM to about 150 μM, about 50 μM to about 100 μM, about 50 μM to about 75 μM, about 75 μM to about 150 PM, about 75 μM to about 100 μM, about 10 μM to about 400 μM, about 10 μM to about 250 PM, about 10 μM to about 200 μM, about 10 μM to about 150 μM, about 10 μM to about 100 PM, about 10 μM to about 50 μM, about 10 μM to about 25 μM, about 15 μM to about 250 μM, about 15 μM to about 200 μM, about 15 μM to about 150 μM, about 15 μM to about 90 μM, about 15 μM to about 50 μM, about 15 μM to about 30 μM, or about 15 μM to about 25 μM. In some embodiments, the admixture comprises the PIC at a concentration of about 145 μM to about 155 PM. In some embodiments, the admixture comprises the PIC at a concentration of about 145 PM, about 146 PM, about 147 μM, about 148 μM, about 149 μM, about 150 μM, about 151 PM, about 152 PM, about 153 μM, about 154 μM, or about 155 μM. In some embodiments, the admixture comprises the PIC at a concentration of about 10 μM, about 15 μM, about 20 μM, about 25 μM, about 30 PM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 110 μM, about 120 μM, about 130 μM, or about 140 μM.

In some embodiments of any of the methods, kits, and compositions provided herein, the admixture of a PAS, a PIC, and a preparation of platelets, the preparation of platelets comprises about 2.0×10¹¹ platelets to about 14.0×10¹¹ platelets. In some embodiments, the admixture comprises at least about 2.0×10¹¹ platelets, at least about 3.0×10¹¹ platelets, at least about 4.0×10¹¹ platelets, at least about 5.0×10¹¹ platelets, at least about 6.0×10¹¹ platelets, at least about 7.0×10¹¹ platelets, at least about 8.0×10¹¹ platelets, at least about 9.0×10¹¹ platelets, at least about 10.0×10¹¹ platelets, at least about 11.0×10¹¹ platelets, or at least about 12.0×10¹¹ platelets. In some embodiments, the preparation of platelets comprises at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets.

In some embodiments, the method of preparing a platelet composition further comprises incubating an admixture of a PAS, a PIC, and a preparation of platelets for a period of about 30 minutes to about 24 hours, wherein incubation is prior to subjecting the admixture to light sufficient to photochemically inactivate a pathogen, if present. The incubation prior to subjecting the admixture to light may be referred to as pre-incubation. In some embodiments, incubating an admixture of a PAS, a PIC, and a preparation of platelets prior to subjecting the admixture to light sufficient to photochemically inactivate a pathogen, if present, is for a period of less than about 24 hours, less than about 22 hours, less than about 20 hours, less than about 18 hours, less than about 16 hours, less than about 14 hours, less than about 12 hours, less than about 10 hours, less than about 8 hours, less than about 6 hours, less than about 5 hours, less than about 4 hours, less than about 3 hours, less than about 2 hours, or less than about 1 hour. In some embodiments, incubating an admixture of a PAS, a PIC, and a preparation of platelets prior to subjecting the admixture to light sufficient to photochemically inactivate a pathogen, if present, is for a period of greater than about 22 hours, greater than about 20 hours, greater than about 18 hours, greater than about 16 hours, greater than about 14 hours, greater than about 12 hours, greater than about 10 hours, greater than about 8 hours, greater than about 6 hours, greater than about 5 hours, greater than about 4 hours, greater than about 3 hours, greater than about 2 hours, greater than about 1 hours, or greater than about 30 minutes. In some embodiments, incubating an admixture of a PAS, a PIC, and a preparation of platelets prior to subjecting the admixture to light sufficient to photochemically inactivate a pathogen, if present, is for a period of about 2 hours, about 4 hours, about 6 hours, about 8 hours, about 10 hours, about 12 hours, about 14 hours, about 16 hours, about 18 hours, about 20 hours, about 22 hours, or about 24 hours. Incubating an admixture of a PAS, a PIC, and a preparation of platelets for a period of about 30 minutes to about 24 hours prior to subjecting the admixture to light sufficient to photochemically inactivate a pathogen may result in an improvement in pathogen inactivation. In some embodiments, such pre-incubation may result in an increase in the degree of inactivation of a pathogen present in the preparation of platelets compared to the degree of inactivation of that pathogen (i.e., same pathogen) resulting from the same method of preparing a platelet composition but without the pre-incubation step. The increase in degree of inactivation of a pathogen may be an increase of at least 1 log, at least 2 logs, at least 3 logs, at least 4 logs, at least 5 logs, at least 6 logs, at least 7 logs, at least 8 logs, at least 9 logs, or at least 10 logs of inactivation of the pathogen. In some embodiments, the pre-incubation may result in an increase in the number of pathogens that are capable of being inactivated if present in the preparation of platelets (e.g., by at least 1, 2, 3, 4, or 5 logs), compared to the number of pathogens that are capable of being inactivated if present in the preparation of platelets as a result of the same method of preparing a platelet composition but without the pre-incubation step. In some embodiments, the improvements in pathogen inactivation described herein are exhibited with respect to one or more bacteria or viruses (e.g., enveloped virus, non-enveloped virus) or parasites.

In some embodiments of any of the methods, kits, and compositions described herein, the wavelength of the light to which the admixture of a PAS, a PIC, and a preparation of platelets wavelength is subjected is between about 200 nm and about 400 nm. In some embodiments, the wavelength of the light is within the ultraviolet A spectrum (e.g., about 315-400 nm). In some embodiments, the duration of the light is between about 1 second and about 30 minutes. In some embodiments, the intensity of the light is between about 1 and about 30 mW/cm². In some embodiments, the dose of the light is between about 1 J/cm² and about 20 J/cm².

In some embodiments of any of the methods described herein, the method is sufficient to inactivate at least 1 log of a pathogen, and the platelet composition after subjecting the admixture of a preparation of platelets and a solution comprising a PAS and a PIC (e.g., admixture of step (b) to light) is suitable for infusion into a subject without further processing to remove residual PIC or photoproducts thereof. In some embodiments, the method is sufficient to inactivate at least 2 logs, at least 3 logs, or at least 4 logs or more of a pathogen, and the platelet composition after subjecting the admixture of a preparation of platelets and a solution comprising a PAS and a PIC (e.g., admixture of step (b)) to light is suitable for infusion into a subject without further processing to remove residual PIC or photoproducts thereof. In some embodiments, a platelet composition suitable for infusion into a subject comprises about 5 μM or less, about 4 μM or less, about 3 μM or less, about 2 μM or less, about 1 μM or less or about 0.5 μM or less of PIC. In some embodiments, a platelet composition suitable for infusion into a subject comprises less than about 5 μM, less than about 4 μM, less than about 3 μM, less than about 2 μM, less than about 1 μM, or less than about 0.5 μM, or less of PIC. In some embodiments of any of the methods described herein, the method is sufficient to inactivate at least 1 log of a pathogen, and the platelet composition after step (c) (e.g., after subjecting the admixture of a preparation of platelets and a solution comprising a PAS and a PIC to light) comprises about 5 μM or less of PIC. In some embodiments, the method is sufficient to inactivate at least 2 logs, at least 3 logs, or at least 4 logs or more of a pathogen, and the platelet composition after step (c) (e.g., after subjecting the admixture of a preparation of platelets and a solution comprising a PAS and a PIC to light) comprises about 4 μM or less, about 3 μM or less, about 2 μM or less, about 1 μM or less or about 0.5 μM or less of PIC. In some embodiments, the platelet composition after step (c) (e.g., after subjecting the admixture of a preparation of platelets and a solution comprising a PAS and a PIC to light) comprises less than 5 PM, less than 4 PM, less than 3 μM, less than 2 μM, less than 1 μM or less than 0.5 μM of PIC. For example, in some embodiments, the method is sufficient to inactivate at least 4 logs a pathogen, and the platelet composition after step (c) (e.g., after subjecting the admixture of a preparation of platelets and a solution comprising a PAS and a PIC to light) comprises about 4 μM or less, about 3 μM or less, about 2 μM or less, about 1 μM or less or about 0.5 μM or less (e.g, less than 5 PM, less than 4 PM, less than 3 μM, less than 2 μM, less than 1 μM, less than 0.5 μM) of PIC. In some embodiments, the concentration of PIC in the admixture of step (b) is at least 10 PM, at least 15 PM, at least 20 μM, at least 25 μM, at least 30 μM, at least 40 μM, at least 50 μM, at least 60 μM, at least 70 μM, at least 80 μM, at least 90 μM, at least 100 μM, at least 110 μM, at least 120 μM, at least 130 μM, at least 140 μM, or at least 150 μM.

In some embodiments, the method of preparing a platelet composition comprises incubating an admixture of a preparation of platelets and a solution comprising a PAS and a PIC, for a period of about 30 minutes to about 24 hours prior to subjecting the admixture to light, wherein: (a) the method is sufficient to inactivate at least 1 log, at least 2 logs, at least 3 logs, at least 4 logs or at least 5 logs or more of a pathogen, if present; (b) the concentration of PIC in the admixture of a PAS, a PIC and a preparation of platelets is about 15 μM to about 150 PM, and (c) the platelet composition after subjecting the admixture of a preparation of platelets and a solution comprising a PAS and a PIC to light comprises less than 5 μM, less than 4 μM, less than 3 μM, less than 2 PM, less than 1 μM or less than 0.5 μM of PIC.

### Platelet Quality

The present disclosure also provides platelet compositions with improved platelet quality suitable for infusion (e.g., infusion into a human subject after pathogen inactivation), wherein the platelet compositions are prepared by any of the methods disclosed herein. For example, platelet compositions prepared by any of the methods disclosed herein retain favorable characteristics (in particular, suitable pH, but also including and not limited to any of dissolved oxygen, carbon dioxide, glucose, lactate, ATP, LDH, p-selectin expression (e.g., CD62P), cellular morphology (e.g., morphology score), extent of shape change or ESC, and hypotonic shock response or HSR) for a longer duration and/or at a level closer to untreated (e.g., non-pathogen-inactivated) platelet compositions during storage after undergoing pathogen inactivation (e.g., as described herein) than is provided with existing methods and processing sets. Such platelet composition characteristics may be those known in the art and commonly measured, such as for example, using assays known in the art.

In some embodiments, the platelet composition prepared by any of the methods disclosed herein retain a pH, even after undergoing pathogen inactivation and storage (e.g., for up to 7 days), closer to the pH of an untreated (e.g., non-pathogen-inactivated) platelet composition or a platelet composition not subjected to storage following pathogen inactivation. In some embodiments, the pH of a platelet composition prepared by any of the methods disclosed herein is ≥6.2, wherein the platelet composition has been stored, following platelet inactivation, at room temperature for at least about 1 day, such as at least about any of 2 days, 3 days, 4 days, 5 days, 6 days, and 7 days. In some embodiments, the pH of a platelet composition prepared by any of the methods disclosed herein is ≥6.4, wherein the platelet composition has been stored, following platelet inactivation, at room temperature for at least about 1 day, such as at least about any of 2 days, 3 days, 4 days, 5 days, 6 days, and 7 days.

### Platelet Units

The present disclosure also provides a platelet composition suitable for infusion (e.g., infusion into a human subject), for example a platelet composition prepared by any of the methods disclosed herein, comprising a minimum number of platelets.

In some embodiments of any of the methods, kits, and compositions provided herein, the platelet composition comprises at least about 2.0×10¹¹ platelets, at least about 3.0×10¹¹ platelets, at least about 4.0×10¹¹ platelets, at least about 5.0×10¹¹ platelets, at least about 6.0×10¹¹ platelets, at least about 7.0×10¹¹ platelets, at least about 8.0×10¹¹ platelets, at least about 9.0×10¹¹ platelets, at least about 10.0×10¹¹ platelets, at least about 11.0×10¹¹ platelets, or at least about 12.0×10¹¹ platelets. In some embodiments, the platelet composition comprises at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets.

In some embodiments, the platelet composition comprises a therapeutic dose (e.g., therapeutic dosage unit) of platelets suitable for infusion into a human subject (e.g., a subject in need of a platelet infusion). In some embodiments, the therapeutic dose comprises a minimum number (e.g., at least a minimum number) of platelets as defined by criteria (e.g., acceptance criteria) of a governmental agency, regulatory agency, institution and/or accrediting organization (e.g., governmental agency, regulatory agency, institution and/or accrediting organization for donated blood products (e.g., donated platelets)). In some embodiments, the regulatory agency is the U.S. Food and Drug Administration (FDA), the European Medicines Agency (EMA), the Australian Therapeutic Goods Administration (TGA), the China Food and Drug Administration (CFDA), or the Japan Ministry of Health, Labour, and Welfare (MHLW). In some embodiments, the accrediting organization is the AABB or the European Directorate for the Quality of Medicines & HealthCare (EDQM). In some embodiments, the platelet composition is prepared in the country of the governmental agency, regulatory agency, institution and/or accrediting organization defining the criteria of a therapeutic dose of platelets. In some embodiments, the therapeutic dosage unit of platelets comprises at least about 2.0×10¹¹ platelets, at least about 2.2×10¹¹ platelets, at least about 2.4×10¹¹, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets, or at least about 3.0×10¹¹ platelets. In some embodiments, the therapeutic dosage unit of platelets comprises at least about 2.4×10¹¹ platelets. In some embodiments, the therapeutic dosage unit of platelets comprises at least about 2.6×10¹¹ platelets. In some embodiments, the therapeutic dosage unit of platelets comprises at least about 3.0×10¹¹ platelets.

In some embodiments, the platelet composition comprises platelets from a plurality of platelet compositions or preparations of platelets. In some embodiments, the platelet composition comprises pooled apheresis-derived platelets from two or more donors, and wherein the pooled apheresis-derived platelets have been treated by any of the methods disclosed herein. In some embodiments, the platelet composition comprises pooled whole blood-derived platelets (e.g., buffy coat platelets, PRP platelets) from two or more donors, and wherein the pooled whole blood-derived platelets have been treated by any of the methods disclosed herein. In some embodiments, the plurality of platelet compositions or preparations of platelets have been treated according to the methods disclosed herein prior to pooling. In some embodiments, the plurality of platelet compositions or preparations of platelets have been treated according to the methods disclosed herein after pooling. In some embodiments, the platelet composition comprises platelets from donors of the same ABO blood type. In some embodiments, the platelet composition comprises platelets from the same ABO and Rh type.

### Storage

In some embodiments of any of the methods, kits, and compositions described herein, the platelet composition may be stored for at least 1, at least 2, at least 3, at least 3, at least 5, at least 6, or at least 7 days, for example on a flatbed agitator (e.g., 60 cycles a minute, model LPR-3, Melco, Glendale, CA, USA) in a temperature-controlled cabinet, at for example, 22±2° C. In some embodiments, the platelet composition may be stored for up to 5, up to 6, or up to 7 days, for example on a flatbed agitator (e.g., 60 cycles a minute, model LPR-3, Melco, Glendale, CA, USA) in a temperature-controlled cabinet, at for example, 22±2° C.

### Platelet Processing

Platelet processing as described in the present disclosure may involve the use of blood product container or blood product bag systems, which are well known in the art. In general, such systems may include more than one plastic container, typically plastic bags, where the bags may be integrally connected with plastic tubing. Some of the containers described herein include such plastic bags as are known in the storage and handling of blood products, including platelet products. Blood bags typically can be designed to hold various volumes of fluid, including, but not limited to, volumes ranging from 50 mL to 2 liters, for example having up to a 350 mL capacity, 450 mL capacity, 500 mL capacity, 1 liter capacity, up to a 1.5 liter capacity, or up to a 2 liter capacity. It is understood that when a method refers to a bag, it includes any such plastic bags used in blood product handling. Where such bags are referred to as “pooling bag”, “mixing bag”, “removal bag”, “product bag”, “storage bag”, or “illumination bag”, it is understood that these bags are typical blood product handling bags, or are similar to such bags in nature. Plastic bags suitable for use according to the present disclosure include for example, those comprising PL2410, as well as other suitable plastics known in the art. Plastic bag materials include polyvinyl chloride, polyolefins, ethylene vinyl acetate, ethylene vinyl acetate blended with other plastics, and the like.

As described herein, where tubing is described as connecting, e.g., two bags, such as for pooling and/or of a processing set, it is understood that the tubing may be joined at some point therebetween by another component of the connection between the two bags. For example, a removal bag connected to a product bag by tubing includes wherein the tubing comprises a filter between the two bags, i.e. the tubing is divided by a filter such that fluid flows from one bag to the other through the tubing and filter. In one example, tubing connecting a removal bag and a product bag can include a filter to remove any loose particles from fluid flowing from the removal device to the product bag, i.e. the tubing is divided by, or interrupted by the filter between the bags. Such filters are designed to remove any small particles that may come off of the removal device, while allowing platelets to pass through the filter. The tubing between bags allows for fluid to flow from one bag to another, which can be blocked to prevent the flow until necessary, e.g. as part of the processing the fluid in one bag may be prevented from flowing to the next bag until required for the next step in a process. As such, an openable seal, such as a clamp, plug, valve or the like is included in or on the tubing connecting the bags, where the clamp, plug, valve or the like can be selectively opened as required, for example to transfer the fluid from one bag to the next. In certain embodiments, the tubing between bags comprises a breakable seal, such as a breakable valve, whereupon breaking the breakable seal allows for the blood product solution to flow between the bags through the tubing. It is understood that the breakable seal is contained within the connection between containers, such that sterility of the system is maintained. It is also understood that a tubing comprising a filter, or a breakable seal, includes where the tubing may be interrupted by the filter or the seal, for example the tubing runs from one bag and is connected to the filter or seal (an incoming portion of the tubing), and the tubing continues from another portion of the filter or seal to another bag (an outgoing portion of the tubing). In such a configuration, fluid flows from the first bag, through the incoming portion of the tubing, through the filter or seal, and through the outgoing portion of the tubing and into the other bag.

Different containers (e.g., bags) within a blood product processing system can be used for different steps of a process. For example, a system of bags to be used for the pathogen inactivation of a preparation of platelets can include one or more of a container with pathogen inactivation compound (PIC) contained within, a container with platelet additive solution (PAS) contained within, a container with PIC and PAS contained within, a container for receiving the preparation of platelets (e.g., platelet donation) and PIC and PAS (e.g. an illumination bag), a bag for the removal of pathogen inactivation compounds and/or by-products thereof from the treated unit of platelets (e.g., referred to as a removal bag, compound adsorption device, CAD), and one or more bags for containing the final platelet composition, e.g., the pathogen inactivated platelet unit (e.g., therapeutic dosage unit) that has the concentration of the inactivating compound and/or by-products thereof reduced to below a desired concentration, which is ready for use or can be stored for later use (e.g., referred to as a product bag, storage bag). Each bag in the system is typically made up of a plastic material. For example, the container for containing a solution of pathogen inactivating compound can be made of a suitable plastic such as PL2411 (Baxter Healthcare), or other plastics such as polyvinyl chloride, polyolefins, ethylene vinyl acetate, ethylene vinyl acetate blended with other plastics, and the like. This container is also overwrapped with a material that is impermeable to light of a wavelength that will activate the photoactive pathogen inactivation compound (for example suitable plastic such as PL2420, Baxter Healthcare). The illumination bag for a photoactivated pathogen inactivating compound requires a clear, durable thermoplastic material that is translucent to light of the selected wavelength. Suitable plastics that are translucent to light in the UVA wavelength range include polyvinyl chloride, polyolefins, ethylene vinyl acetate, ethylene vinyl acetate blended with other plastics, or other blends of thermoplastic polymers. Such suitable plastics include PL2410 (Baxter Healthcare) and PL732 (Baxter Healthcare). Similar materials may be used to make the removal bag and the product bag. The product bags include, for example, those made of PL2410. Suitable bag materials are discussed, for example, in PCT publication number WO 2003078023, and U.S. Pat. No. 7,025,877, the disclosures of which are hereby incorporated by reference as it relates to such bag materials and related materials. In all cases, the materials used in preparing the processing set have to be sterilizable by known methods such as steam and gamma or electron beam radiation used to ensure sterility of the processing set. While these are exemplary materials for making the bags, the methods, kits, and compositions described herein are applicable to processes using any suitable bag material as would be readily available to one skilled in the art, and can also be used with containers other than bags. The bags used for illumination, removal, and storage are also designed to allow for gases such as oxygen and carbon dioxide to go into and out of the blood bag, so that the platelets therein have adequate oxygen supply and carbon dioxide levels during the processing and storage.

Certain aspects of the present disclosure relate to processing sets. The processing sets of the present disclosure may find use, inter alia, in preparing a plurality of platelet compositions (e.g., platelet units) suitable for infusion, e.g., as described herein. Any of the exemplary components such as bags and tubings described supra may find use in the processing sets of the present disclosure.

### Pathogen Inactivation

Blood products, including platelet-containing blood products, may contain pathogens, or may be contaminated with pathogens during processing. As such, it is desirable to subject such blood products to a pathogen inactivation process in order to reduce the risk of transfusion-transmitted diseases. Various processes and methods have been assessed to mitigate the risk of transfusion-associated disease transmission in platelet-containing blood products. Aside from screening and detection of pathogens and subsequent elimination of contaminated blood products, processes that incorporate treatments to inactivate pathogens (i.e., pathogen inactivation) that may be present are available. Ideally, such a process results in the inactivation of a broad range of pathogens such as viruses, bacteria and parasites that may be present in the blood product. In certain embodiments, the methods of pathogen inactivation require addition of an amount of pathogen inactivating compound to a preparation of platelets (e.g., treating the platelet preparation). For example, pathogen inactivation may involve the addition of a low molecular weight compound that inactivates various pathogens, where a particular method involves the addition of a photosensitizer that, when activated by illumination using light of suitable wavelengths, will inactivate a variety of pathogens that may be present. Two methods that are commercially available include the addition of amotosalen or riboflavin to the platelets, with subsequent illumination with UV light. Other methods include illumination with UV light without addition of a photosensitizer, as well as illumination with other photoactive compounds, including psoralen derivatives other than amotosalen, isoalloxazines other than riboflavin, alloxazines, dyes such as phthalocyanines, phenothiazine dyes (e.g. methylene blue, azure B, azure C, thionine, toluidine blue), porphyrin derivatives (e.g. dihematoporphyrin ether, hematoporphyrin derivatives, benzoporphyrin derivatives, alkyl-substituted sapphyrin), and merocyanine 540 (Prodouz et al., Blood Cells 1992, 18(1):101-14; Sofer, Gail, BioPharm, August 2002). Other pathogen inactivation systems include, for example, those described in PCT publication numbers WO 2012071135; WO 2012018484; WO 2003090794; WO 2003049784; WO 1998018908; WO 1998030327; WO 1996008965; WO 1996039815; WO 1996039820; WO 1996040857; WO 1993000005; US patent application number US 20050202395; and U.S. Pat. Nos. 8,296,071 and 6,548,242, the disclosures of which are hereby incorporated by reference as they relate to pathogen inactivation in blood products. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen. Where addition of a compound to the platelets is used for pathogen inactivation, whether the method requires illumination or not, in some instances it is desirable to remove any residual pathogen inactivation compound or by-product (e.g., photoproduct) thereof.

Methods for pathogen inactivation and removal of pathogen inactivating compound as described herein are applicable to any platelet preparations, whether the platelet preparations comprise individual platelet donations (e.g., apheresis collected platelets) or pooled platelet preparations.

Some pathogen inactivation methods disclosed herein may not require the use of a removal device (i.e., a device for reducing the concentration of pathogen inactivation compound, such as a small organic compound, and by-products thereof in a preparation of platelets), while substantially maintaining a desired biological activity of the platelets.

Some pathogen inactivation methods may require the use of a removal device(i.e., a device for reducing the concentration of pathogen inactivation compound, such as a small organic compound, and by-products thereof in a preparation of platelets), while substantially maintaining a desired biological activity of the platelets. In some embodiments, the removal device is referred to as a compound adsorption device (CAD), and may comprise a container (e.g., CAD container, CAD bag) containing one or more materials, such as for example, adsorbent particles, and which is suitable for also containing a preparation of platelets from which the concentration of pathogen inactivation compound and by-products thereof are to be reduced. Such a removal device is generally intended to be used in a batch mode, i.e. the device is placed in contact with the platelets, and continued contact with the removal device, e.g. with shaking to allow essentially the entirety of the solution of platelets to come into contact with the removal device over time of contact, results in reducing the levels of pathogen inactivation compound. Such batch devices entail the use of an adsorbent particle that binds the pathogen inactivation compound, and can be used by either adding adsorbent particles directly to the platelet container (e.g., bag) following illumination or transferring the platelets to a bag containing the adsorbent particles following illumination and the platelets are then agitated for a specified period of time with the platelet preparations contacting the removal device. While free adsorbent particles may be used as a removal device, such particles may be contained within a mesh pouch, such as a polyester or nylon mesh pouch, which allows for contact of the platelet solution with the adsorbent particles while containing the particles within the pouch. Alternatively, the adsorbent particles may be immobilized within a matrix, where the immobilized matrix can reside directly in the blood bag used for batch removal, or may be similarly contained within a mesh pouch. In some instances, the removal device comprises porous adsorbent particles in an amount sufficient to reduce the pathogen inactivation compound to below a desired concentration, wherein the adsorbent particles have an affinity for the pathogen inactivation compound, where it is understood such adsorbent particle can be selected to best adsorb the compound or compounds to be removed, with minimal effect on components that should not be removed or damaged by contact with the adsorbent particle. A variety of adsorbent particles are known, including generally particles made from any natural or synthetic material capable of interacting with compounds to be removed, including particulates made of natural materials such as activated carbon, silica, diatomaceous earth, and cellulose, and synthetic materials such as hydrophobic resins, hydrophilic resins or ion exchange resins. Such synthetic resins include, for example, carbonaceous materials, polystyrene, polyacrylic, polyacrylic ester, cation exchange resin, and polystyrene-divinylbenzene. Detailed description of such removal devices suitable for use in the methods as described herein can be found in PCT publication numbers WO 1996040857, WO 1998030327, WO 1999034914, and WO 2003078023, the disclosures of which are hereby incorporated by reference with respect to the discussion of such removal devices and the adsorbent particles and other materials used to prepare such devices. Exemplary adsorbent particles include, but are not limited to, Amberlite (Rohm and Haas) XAD-2, XAD-4, XAD-7, XAD-16, XAD-18, XAD-1180, XAD-1600, XAD-2000, XAD-2010; Amberchrom (Toso Haas) CG-71m, CG-71c, CG-161m, CG161c; Diaion Sepabeads (Mitsubishi Chemicals) HP20, SP206, SP207, SP850, HP2MG, HP20SS, SP20MS; Dowex (Dow Chemical) XUS-40285, XUS-40323, XUS-43493 (also referred to as Optipore V493 (dry form) or Optipore L493 (hydrated form)), Optipore V503, Optipore SD-2; Hypersol Macronet (Purolite) MN-100, MN-102, MN-150, MN-152, MN-170, MN-200, MN-202, MN-250, MN-252, MN-270, MN-300, MN-400, MN-500, MN-502, Purosorb (Purolite) PAD 350, PAD 400, PAD 428, PAD 500, PAD 550, PAD 600, PAD 700, PAD 900, and PAD 950. The material used to form the immobilized matrix comprises a low melting polymer, such as nylon, polyester, polyethylene, polyamide, polyolefin, polyvinyl alcohol, ethylene vinyl acetate, or polysulfone. In one example, the adsorbent particles immobilized in a matrix are in the form of a sintered medium. While it is understood that the methods, kits, and compositions described herein may encompass removal devices as are known in the art, such methods and devices may be exemplified using the removal device of an amotosalen inactivated platelet product as is commercially available. Such a removal device comprises Hypersol Macronet MN-200 adsorbent contained within a sintered matrix, where the sintered matrix comprises PL2410 plastic as a binder. In one instance, the removal device comprises Hypersol Macronet MN-200 adsorbent in a sintered matrix comprising PL2410, wherein the Hypersol Macronet MN-200 is in an amount of about 5-50 grams, about 5-10 grams, about 10-15 grams, about 15-20 grams, about, 20-25 grams, about 25-30 grams, about 30-35 grams, about 35-40 grams, about 40-45 grams or about 45-50 grams dry weight equivalent.

As various resins may require different processing when used to make the removal devices useful in the methods, kits, and compositions as described herein, comparison of amounts of adsorbent resins described herein, unless otherwise indicated, are comparison of the dry weight of the resin. For example, the resins are dried to <5% water prior to processing, and the equivalent of the dry weight of adsorbent is used in comparing amounts of resin in use. For example, Hypersol Macronet MN-200 is processed to stabilize the adsorbent, or what is typically referred to as wetting the adsorbent, so as to be directly usable upon contact with a platelet unit. Such a wetted sample may include, for example, about 50% glycerol or other suitable wetting agent. In some embodiments, the adsorbent resin is a polystyrene-divinylbenzene resin. In some embodiments, the polystyrene-divinylbenzene resin is Hypersol Macronet MN-200. In some embodiments, the adsorbent is contained within a sintered matrix, wherein the sintered matrix comprises PL2410 binder. In some embodiments, Hypersol Macronet MN-200 adsorbent is contained within a sintered matrix to provide a removal device.

In some embodiments of any of the methods, kits, and compositions described herein, one or more component (e.g., container, CAD, PIC) may be derived from or substantially similar to a commercially available pathogen inactivation system, such as for example the INTERCEPT® Blood System (Cerus). The INTERCEPT® Blood System is well known in the art as a system for pathogen inactivation, with widespread adoption in European blood centers and FDA approval in the United States. For greater description of the INTERCEPT® Blood System and pathogen inactivation methods and compositions related thereto, see, e.g., U.S. Pat. Nos. 5,399,719, 5,556,993, 5,578,736, 5,585,503, 5,593,823, 5,625,079, 5,654,443, 5,712,085, 5,871,900, 5,972,593, 6,004,741, 6,004,742, 6,017,691, 6,194,139, 6,218,100, 6,503,699, 6,544,727, 6,951,713, 7,037,642, and 7,611,831; and PCT publication numbers WO 1995000141, WO 1996014739, WO 1997021346, WO 1998030327, WO 1999034914, and WO1999034915, the disclosures of each of which are hereby incorporated by reference as they relate to pathogen inactivation in blood products.

### Kits for Preparing a Platelet Composition

The present disclosure, provides, in some aspects, kits, e.g., processing sets, for preparing a platelet composition according to any of the methods disclosed herein. In some embodiments, the kit is a disposable processing set.

In some embodiments, the kit comprises (a) a first container comprising a solution comprising a platelet additive solution (PAS) and a pathogen inactivation compound (PIC), and (b) instructions for use in preparing a platelet composition.

The kits for preparing a platelet composition (e.g., pathogen inactivated platelet composition) disclosed herein comprise a solution comprising a platelet additive solution (PAS) and a pathogen inactivation compound (PIC), wherein the solution comprising the PAS and the PIC is of a sufficient volume for preparing any number of platelet compositions (e.g., platelet unit or therapeutic dose). In some embodiments, the kit comprises two or more first containers, wherein each of the two or more first containers contains a different volume of a solution comprising a platelet additive solution (PAS) and a pathogen inactivation compound (PIC), and wherein one of the two or more first containers may be selected for use based on the amount of the number or volume of platelet compositions to be prepared. In some embodiments the kit comprises three first containers, wherein one first container contains a sufficient volume of a solution comprising a platelet additive solution (PAS) and a pathogen inactivation compound (PIC) for preparing one platelet composition (e.g., platelet unit, therapeutic dose), another first container contains a sufficient volume of a solution comprising a platelet additive solution (PAS) and a pathogen inactivation compound (PIC) for preparing two platelet compositions, and yet another first container contains a sufficient volume of a solution comprising a platelet additive solution (PAS) and a pathogen inactivation compound (PIC) for preparing three platelet compositions. When using this kit in any of the methods provided herein, one of the three first containers may be selected for use based on the number of platelet compositions to be prepared.

In some embodiments, the kit for preparing a platelet composition comprises: (a) a first container comprising a solution comprising a platelet additive solution (PAS) and a pathogen inactivation compound (PIC), and (b) a second container suitable for containing a preparation of platelets in admixture with the solution comprising the PAS and the PIC, wherein the first container is not coupled to the second container. In some embodiments, the first container is suitable for admixing a preparation of platelets with a solution comprising a PAS and a PIC. In some embodiments, the first container is suitable for subjecting a preparation of platelets in admixture with a solution comprising a PAS and a PIC to light sufficient to photochemically inactivate a pathogen, if present. In some embodiments, the first container is made of a material that is substantially translucent to light in the photochemical inactivation wavelength range (e.g., about 200 nm to about 400 nm, ultraviolet A spectrum). In some embodiments, the second container comprises a compound adsorption device (CAD). In some embodiments, the second container is suitable for storing a platelet composition. In some embodiments, the first container is configured to be integrally connected to the second container (e.g., by a flexible plastic tube). In some embodiments, the first container is configured to be sterilely coupled to the second container. In some embodiments, the kit further comprises one or more components (e.g., tubing, flexible plastic tubing) for connecting the first container to the second container. In some embodiments, the kit for preparing a platelet composition further comprises at least one (e.g., 1, 2, or 3) storage container, wherein the at least one storage container is suitable for storing a platelet composition, and wherein the at least one storage container is coupled to the second container. In some embodiments, the at least one storage container is integrally connected to the second container (e.g., by a flexible plastic tube). In some embodiments, the at least one storage container is sealed but has an openable flow path to the second container. In some embodiments, the at least one storage container is sterilely coupled to the second container. In some embodiments, the first container is suitable for connecting to an apheresis device or to a container containing a preparation of platelets.

In some embodiments, the kit for preparing a platelet composition comprises: (a) a first container comprising a solution comprising a platelet additive solution (PAS) and a pathogen inactivation compound (PIC), and (b) a second container suitable for containing a preparation of platelets in admixture with the solution comprising the PAS and the PIC, wherein the first container is not coupled to the second container. In some embodiments, the first container is suitable for admixing a preparation of platelets with a solution comprising a PAS and a PIC. In some embodiments, the second container is suitable for admixing a preparation of platelets with a solution comprising a PAS and a PIC. In some embodiments, the second container is suitable for subjecting a preparation of platelets in admixture with a solution comprising a PAS and a PIC to light sufficient to photochemically inactivate a pathogen, if present. In some embodiments, the second container is made of a material that is substantially translucent to light in the photochemical inactivation wavelength range (e.g., about 200 nm to about 400 nm, ultraviolet A spectrum). In some embodiments, the second container comprises a compound adsorption device (CAD). In some embodiments, the second container is suitable for storing a platelet composition. In some embodiments, the first container is configured to be integrally connected to the second container (e.g., by a flexible plastic tube). In some embodiments, the first container is configured to be sterilely coupled to the second container. In some embodiments, the kit further comprises one or more components (e.g., tubing, flexible plastic tubing) for connecting the first container to the second container. In some embodiments, the kit for preparing a platelet composition further comprises at least one (e.g., 1, 2, or 3) storage container, wherein the at least one storage container is suitable for storing a platelet composition, and wherein the at least one storage container is coupled to the second container. In some embodiments, the at least one storage container is integrally connected to the second container (e.g., by a flexible plastic tube). In some embodiments, the at least one storage container is sealed but has an openable flow path to the second container. In some embodiments, the at least one storage container is sterilely coupled to the second container. In some embodiments, the first container is suitable for connecting to an apheresis device or to a container containing a preparation of platelets.

In some embodiments, the kit for preparing a platelet composition comprises: (a) a first container comprising a solution comprising a platelet additive solution (PAS) and a pathogen inactivation compound (PIC), and (b) a second container suitable for containing a preparation of platelets in admixture with the solution comprising the PAS and the PIC, wherein the first container is not coupled to the second container. In some embodiments, the first container is suitable for admixing a preparation of platelets with a solution comprising a PAS and a PIC. In some embodiments, the second container is suitable for admixing a preparation of platelets with a solution comprising a PAS and a PIC. In some embodiments, the second container is suitable for subjecting a preparation of platelets in admixture with a solution comprising a PAS and a PIC to light sufficient to photochemically inactivate a pathogen, if present. In some embodiments, the second container is made of a material that is substantially translucent to light in the photochemical inactivation wavelength range (e.g., about 200 nm to about 400 nm, ultraviolet A spectrum). In some embodiments, the first container is configured to be integrally connected to the second container (e.g., by a flexible plastic tube). In some embodiments, the first container is configured to be sterilely coupled to the second container. In some embodiments, the kit further comprises one or more components (e.g., tubing, flexible plastic tubing) for connecting the first container to the second container. In some embodiments, the kit for preparing a platelet composition further comprises a third container, wherein the third container comprises a compound adsorption device (CAD), and wherein the third container is coupled to the second container. In some embodiments, the third container is integrally connected to the second container (e.g., by a flexible plastic tube). In some embodiments, the third container is sealed but has an openable flow path to the second container. In some embodiments, the third container is sterilely coupled to the second container. In some embodiments, the third container is suitable for storing a platelet composition. In some embodiments, the kit for preparing a platelet composition further comprises at least one (e.g., 1, 2, or 3) storage container, wherein the at least one storage container is suitable for storing a platelet composition, and wherein the at least one storage container is coupled to the third container. In some embodiments, the at least one storage container is integrally connected to the third container (e.g., by a flexible plastic tube). In some embodiments, the at least one storage container is sealed but has an openable flow path to the third container. In some embodiments, the at least one storage container is sterilely coupled to the third container. In some embodiments, the first container is suitable for connecting to an apheresis device or to a container containing a preparation of platelets.

In some embodiments, the kit for preparing a platelet composition comprises: (a) a first container comprising a solution comprising a platelet additive solution (PAS) and a pathogen inactivation compound (PIC), and (b) a second container suitable for containing a preparation of platelets in admixture with the solution comprising the PAS and the PIC, wherein the first container is not coupled to the second container. In some embodiments, the second container is suitable for admixing a preparation of platelets with a solution comprising a PAS and a PIC. In some embodiments, the second container is suitable for subjecting a preparation of platelets in admixture with a solution comprising a PAS and a PIC to light sufficient to photochemically inactivate a pathogen, if present. In some embodiments, the second container is made of a material that is substantially translucent to light in the photochemical inactivation wavelength range (e.g., about 200 nm to about 400 nm, ultraviolet A spectrum). In some embodiments, the second container comprises a compound adsorption device (CAD). In some embodiments, the second the second container is suitable for storing a platelet composition. In some embodiments, the first container is configured to be integrally connected to the second container (e.g., by a flexible plastic tube). In some embodiments, the first container is configured to be sterilely coupled to the second container. In some embodiments, the kit further comprises one or more components (e.g., tubing, flexible plastic tubing) for connecting the first container to the second container. In some embodiments, the kit for preparing a platelet composition further comprises at least one (e.g., 1, 2, or 3) storage container, wherein the at least one storage container is suitable for storing a platelet composition, and wherein the at least one storage container is coupled to the second container. In some embodiments, the at least one storage container is integrally connected to the second container (e.g., by a flexible plastic tube). In some embodiments, the at least one storage container is sealed but has an openable flow path to the second container. In some embodiments, the at least one storage container is sterilely coupled to the second container. In some embodiments, the second container is suitable for connecting to an apheresis device or to a container containing a preparation of platelets.

In some embodiments, the kit for preparing a platelet composition comprises: (a) a first container comprising a solution comprising a platelet additive solution (PAS) and a pathogen inactivation compound (PIC), and (b) a second container suitable for containing a preparation of platelets in admixture with the solution comprising the PAS and the PIC, wherein the first container is not coupled to the second container. In some embodiments, the second container is suitable for admixing a preparation of platelets with a solution comprising a PAS and a PIC. In some embodiments, the second container is suitable for subjecting a preparation of platelets in admixture with a solution comprising a PAS and a PIC to light sufficient to photochemically inactivate a pathogen, if present. In some embodiments, the second container is made of a material that is substantially translucent to light in the photochemical inactivation wavelength range (e.g., about 200 nm to about 400 nm, ultraviolet A spectrum). In some embodiments, the first container is configured to be integrally connected to the second container (e.g., by a flexible plastic tube). In some embodiments, the first container is configured to be sterilely coupled to the second container. In some embodiments, the kit further comprises one or more components (e.g., tubing, flexible plastic tubing) for connecting the first container to the second container. In some embodiments, the kit for preparing a platelet composition further comprises a third container, wherein the third container comprises a compound adsorption device (CAD), and wherein the third container is coupled to the second container. In some embodiments, the third container is integrally connected to the second container (e.g., by a flexible plastic tube). In some embodiments, the third container is sealed but has an openable flow path to the second container. In some embodiments, the third container is sterilely coupled to the second container. In some embodiments, the third container is suitable for storing a platelet composition. In some embodiments, the kit for preparing a platelet composition further comprises at least one (e.g., 1, 2, or 3) storage container, wherein the at least one storage container is suitable for storing a platelet composition, and wherein the at least one storage container is coupled to the third container. In some embodiments, the at least one storage container is integrally connected to the third container (e.g., by a flexible plastic tube). In some embodiments, the at least one storage container is sealed but has an openable flow path to the third container. In some embodiments, the at least one storage container is sterilely coupled to the third container. In some embodiments, the second container is suitable for connecting to an apheresis device or to a container containing a preparation of platelets.

In some embodiments, the kit for preparing a platelet composition, comprises (a) a first container comprising a platelet additive solution (PAS), (b) a second container comprising a pathogen inactivation compound (PIC), and (c) a third container suitable for containing a preparation of platelets in admixture with the with the PAS and the PIC, wherein the first and second containers are coupled to one another, and wherein neither of the first and second containers is coupled to the third container. In some embodiments, the first container and the second container are configured to have a sealed but openable flow path between each other. In some embodiments, the first container is configured to be integrally connected to the third container (e.g., by a flexible plastic tube). In some embodiments, the first container is configured to be sterilely coupled to the third container. In some embodiments, the kit further comprises one or more components (e.g., tubing, flexible plastic tubing) for connecting a first container to the third container. In some embodiments, the second container is configured to be integrally connected to the third container (e.g., by a flexible plastic tube). In some embodiments, the second container is configured to be sterilely coupled to the third container. In some embodiments, the kit further comprises one or more components (e.g., tubing, flexible plastic tubing) for connecting the second container to the third container. In some embodiments, the first container is suitable for combining the PAS with the PIC. In some embodiments, the second container is suitable for combining the PAS with the PIC. Any one or more of the first, second, and third containers may be suitable for admixing a preparation of platelets with a solution comprising a PAS and a PIC. In some embodiments, the first container is suitable for subjecting a preparation of platelets in admixture with a solution comprising a PAS and a PIC to light sufficient to photochemically inactivate a pathogen, if present. In some embodiments, the second container is suitable for subjecting a preparation of platelets in admixture with a solution comprising a PAS and a PIC to light sufficient to photochemically inactivate a pathogen, if present. In some embodiments, the third container is suitable for subjecting a preparation of platelets in admixture with a solution comprising a PAS and a PIC to light sufficient to photochemically inactivate a pathogen, if present. Any one or more of the first, second, and third containers may be made of a material that is substantially translucent to light in the photochemical inactivation wavelength range (e.g., about 200 nm to about 400 nm, ultraviolet A spectrum). In some embodiments, the third container comprises a compound adsorption device (CAD). In some embodiments, the third container is suitable for storing a platelet composition. In some embodiments, the kit for preparing a platelet composition further comprises at least one (e.g., 1, 2, or 3) storage container, wherein the at least one storage container is suitable for storing a platelet composition, and wherein the at least one storage container is coupled to the third container. In some embodiments, the at least one storage container is integrally connected to the third container (e.g., by a flexible plastic tube). In some embodiments, the at least one storage container is sealed but has an openable flow path to the third container. In some embodiments, the at least one storage container is sterilely coupled to the third container. Any one or more of the first, second, and third containers may be suitable for connecting to an apheresis device or to a container containing a preparation of platelets.

In some embodiments, the kit for preparing a platelet composition, comprises (a) a first container comprising a platelet additive solution (PAS), (b) a second container comprising a pathogen inactivation compound (PIC), and (c) a third container suitable for containing a preparation of platelets in admixture with the with the PAS and the PIC, wherein the first and second containers are coupled to one another, and wherein neither of the first and second containers is coupled to the third container. In some embodiments, the first container and the second container are configured to have a sealed but openable flow path between each other. In some embodiments, the first container is configured to be integrally connected to the third container (e.g., by a flexible plastic tube). In some embodiments, the first container is configured to be sterilely coupled to the third container. In some embodiments, the kit further comprises one or more components (e.g., tubing, flexible plastic tubing) for connecting a first container to the third container. In some embodiments, the second container is configured to be integrally connected to the third container (e.g., by a flexible plastic tube). In some embodiments, the second container is configured to be sterilely coupled to the third container. In some embodiments, the kit further comprises one or more components (e.g., tubing, flexible plastic tubing) for connecting the second container to the third container. In some embodiments, the first container is suitable for combining the PAS with the PIC. In some embodiments, the second container is suitable for combining the PAS with the PIC. Any one or more of the first, second, and third containers may be suitable for admixing a preparation of platelets with a solution comprising a PAS and a PIC. In some embodiments, the first container is suitable for subjecting a preparation of platelets in admixture with a solution comprising a PAS and a PIC to light sufficient to photochemically inactivate a pathogen, if present. In some embodiments, the second container is suitable for subjecting a preparation of platelets in admixture with a solution comprising a PAS and a PIC to light sufficient to photochemically inactivate a pathogen, if present. In some embodiments, the third container is suitable for subjecting a preparation of platelets in admixture with a solution comprising a PAS and a PIC to light sufficient to photochemically inactivate a pathogen, if present. Any one or more of the first, second, and third containers may be made of a material that is substantially translucent to light in the photochemical inactivation wavelength range (e.g., about 200 nm to about 400 nm, ultraviolet A spectrum). In some embodiments, the kit for preparing a platelet composition further comprises a fourth container, wherein the fourth container comprises a compound adsorption device (CAD). In some embodiments, the fourth container is configured to be integrally connected to the third container (e.g., by a flexible plastic tube). In some embodiments, the fourth container is configured to have an openable flow path to the third container. In some embodiments, the fourth container is configured to be sterilely coupled to the third container. In some embodiments, the kit further comprises one or more components (e.g., tubing, flexible plastic tubing) for connecting the fourth container to the third container. In some embodiments, the fourth container is suitable for storing a platelet composition. In some embodiments, the kit for preparing a platelet composition further comprises at least one (e.g., 1, 2, or 3) storage container, wherein the at least one storage container is suitable for storing a platelet composition, and wherein the at least one storage container is coupled to the fourth container. In some embodiments, the at least one storage container is integrally connected to the fourth container (e.g., by a flexible plastic tube). In some embodiments, the at least one storage container is sealed but has an openable flow path to the fourth container. In some embodiments, the kit for preparing a platelet composition further comprises at least one (e.g., 1, 2, or 3) storage container, wherein the at least one storage container is suitable for storing a platelet composition, and wherein the at least one storage container is coupled to the fourth container. Any one or more of the first, second, and third containers may be suitable for connecting to an apheresis device or to a container containing a preparation of platelets.

In some embodiments, the kit for preparing a platelet composition, comprises (a) a first container comprising a platelet additive solution (PAS), (b) a second container comprising a pathogen inactivation compound (PIC), and (c) a third container suitable for containing a preparation of platelets in admixture with the with the PAS and the PIC, wherein neither of the first and second containers is coupled to the third container. In some embodiments, the first container and the second container are configured to be coupled (e.g., sterilely coupled) to one another. In some embodiments, the kit further comprises one or more components (e.g., tubing, flexible plastic tubing) for connecting the first container to the second container In some embodiments, the first container is configured to be integrally connected to the third container (e.g., by a flexible plastic tube). In some embodiments, the first container is configured to be sterilely coupled to the third container. In some embodiments, the kit further comprises one or more components (e.g., tubing, flexible plastic tubing) for connecting a first container to the third container. In some embodiments, the second container is configured to be integrally connected to the third container (e.g., by a flexible plastic tube). In some embodiments, the second container is configured to be sterilely coupled to the third container. In some embodiments, the kit further comprises one or more components (e.g., tubing, flexible plastic tubing) for connecting the second container to the third container. In some embodiments, the first container is suitable for combining the PAS with the PIC. In some embodiments, the second container is suitable for combining the PAS with the PIC. Any one or more of the first, second, and third containers may be suitable for admixing a preparation of platelets with a solution comprising a PAS and a PIC. In some embodiments, the first container is suitable for subjecting a preparation of platelets in admixture with a solution comprising a PAS and a PIC to light sufficient to photochemically inactivate a pathogen, if present. In some embodiments, the second container is suitable for subjecting a preparation of platelets in admixture with a solution comprising a PAS and a PIC to light sufficient to photochemically inactivate a pathogen, if present. In some embodiments, the third container is suitable for subjecting a preparation of platelets in admixture with a solution comprising a PAS and a PIC to light sufficient to photochemically inactivate a pathogen, if present. Any one or more of the first, second, and third containers may be made of a material that is substantially translucent to light in the photochemical inactivation wavelength range (e.g., about 200 nm to about 400 nm, UVA spectrum). In some embodiments, the third container comprises a compound adsorption device (CAD). In some embodiments, the third container is suitable for storing a platelet composition. In some embodiments, the kit for preparing a platelet composition further comprises at least one (e.g., 1, 2, or 3) storage container, wherein the at least one storage container is suitable for storing a platelet composition, and wherein the at least one storage container is coupled to the third container. In some embodiments, the at least one storage container is integrally connected to the third container (e.g., by a flexible plastic tube). In some embodiments, the at least one storage container is sealed but has an openable flow path to the third container. In some embodiments, the at least one storage container is sterilely coupled to the third container. Any one or more of the first, second, and third containers may be suitable for connecting to an apheresis device or to a container containing a preparation of platelets.

In some embodiments of any of the kits described herein, the solution of the PAS and the PIC has a volume of between about 10 mL and about 1000 mL. In some embodiments, the solution of the PAS and the PIC has a volume of between about 200 mL and about 900 mL, between about 300 mL and about 800 mL, between about 400 mL and about 700 mL, or between about 500 mL and about 600 mL. In some embodiments, the solution of the PAS and the PIC has a volume of about 100 mL, about 200 mL, about 300 mL, about 400 mL, about 500 mL, about 600 mL, about 700 mL, about 800 mL, about 900 mL, or about 1000 mL. In some embodiments, the solution of the PAS and the PIC has a volume of less than about 1000 mL, less than about 800 mL, less than about 600 mL, less than about 500 mL, less than about 400 mL, less than about 300 mL, less than about 200 mL, less than about 100 mL, or less than about 50 mL. In some embodiments, the solution of the PAS and the PIC has a volume of greater than about 800 mL, greater than about 600 mL, greater than about 500 mL, greater than about 400 mL, greater than about 300 mL, greater than about 200 mL, greater than about 100 mL, greater than about 50 mL, or greater than about 10 mL.

In some embodiments of any of the kits described herein, the concentration of PIC in the solution of the PAS and the PIC is about 25 μM to about 1200 μM, about 50 μM to about 1000 PM, about 50 μM to about 750 μM, about 50 μM to about 500 μM, about 75 μM to about 500 PM, about 100 μM to about 400 μM, about 150 μM to about 350 μM, about 200 μM to about 300 PM, or about 225 μM to about 250 μM. In some embodiments, the concentration of PIC is about 25 PM, about 50 μM, about 75 μM, about 100 μM, about 125 μM, about 150 μM, about 175 μM, about 200 μM, about 250 μM about 275 μM, about 300 μM, about 325 μM, about 350 μM, about 375 μM, about 400 μM, about 450 μM, about 500 μM, about 550 μM, about 600 μM, about 650 μM, about 700 μM, about 750 μM, about 800 μM, about 850 μM, about 900 μM, about 1000 μM, about 1100 μM, about 1200 μM, about 1300 μM, about 1400 μM, or about 1500 μM. In some embodiments, the concentration of PIC is about 225 μM to about 235 μM. In some embodiments, the concentration of PIC is about 225 μM, about 226 μM, about 227 μM, about 228 PM, about 229 PM, about 230 μM, about 231 μM, about 232 μM, about 233 μM, about 234 PM, or about 235 PM.

In some embodiments of any of the kits described herein, the PIC is a psoralen. In some embodiments of any of the kits described herein, the PIC is amotosalen. In some embodiments of any of the kits described herein, the PIC is selected from the group consisting of a isoalloxazine, an alloxazine, a phthalocyanine, a phenothiazine, a porphyrin, merocyanine 540, and salts or free bases thereof.

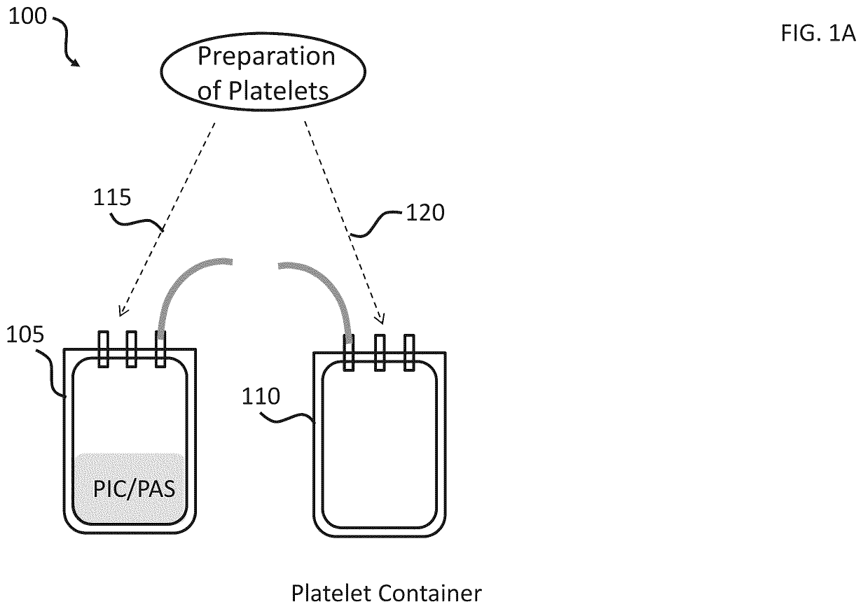

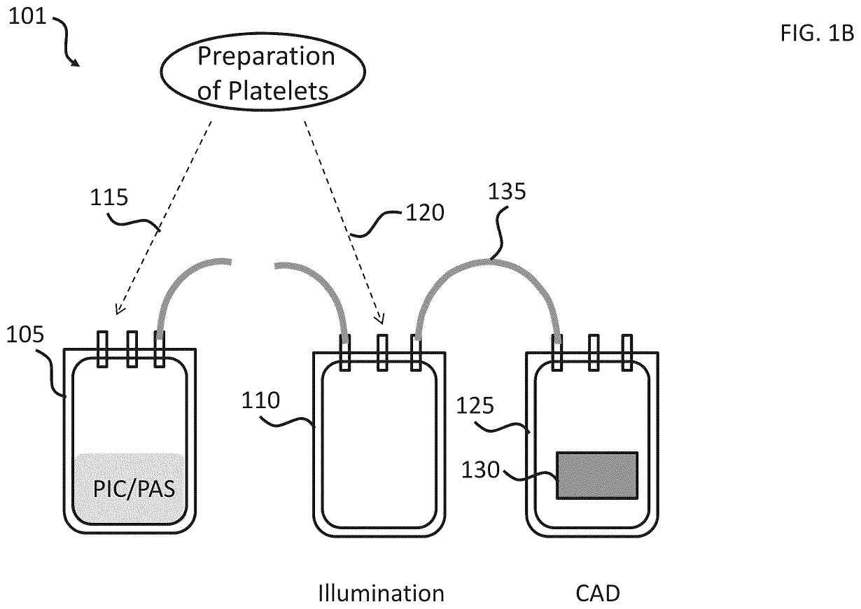

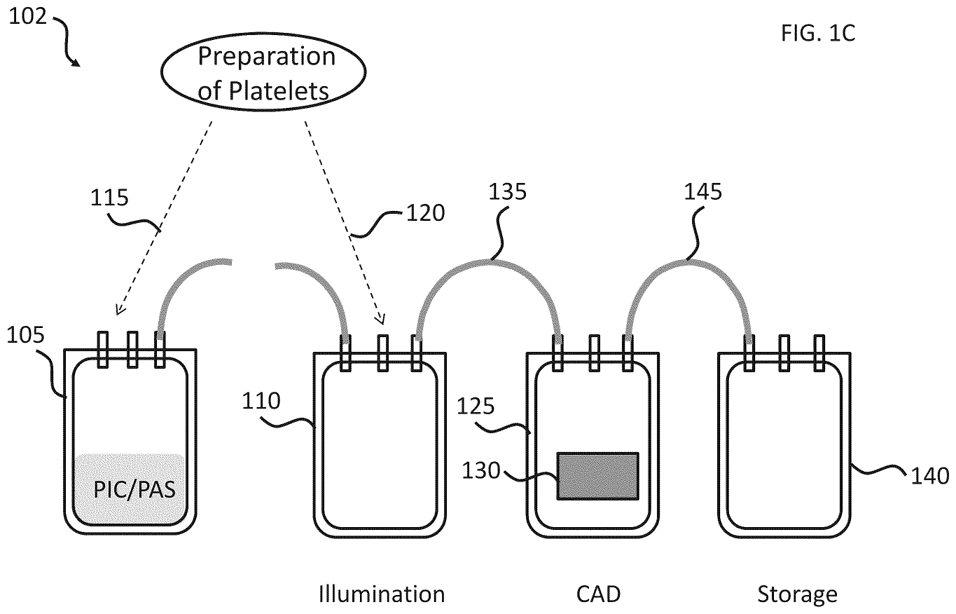

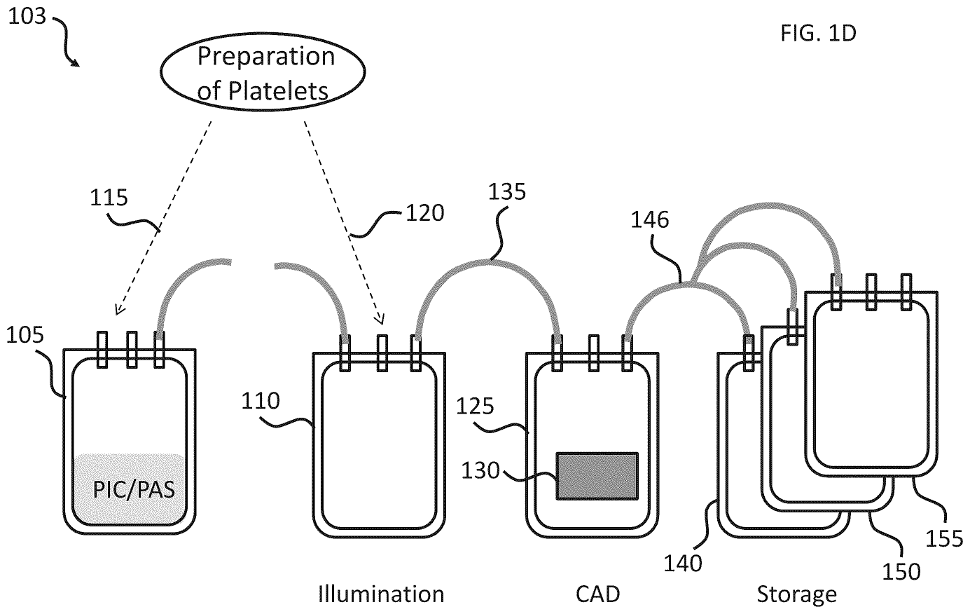

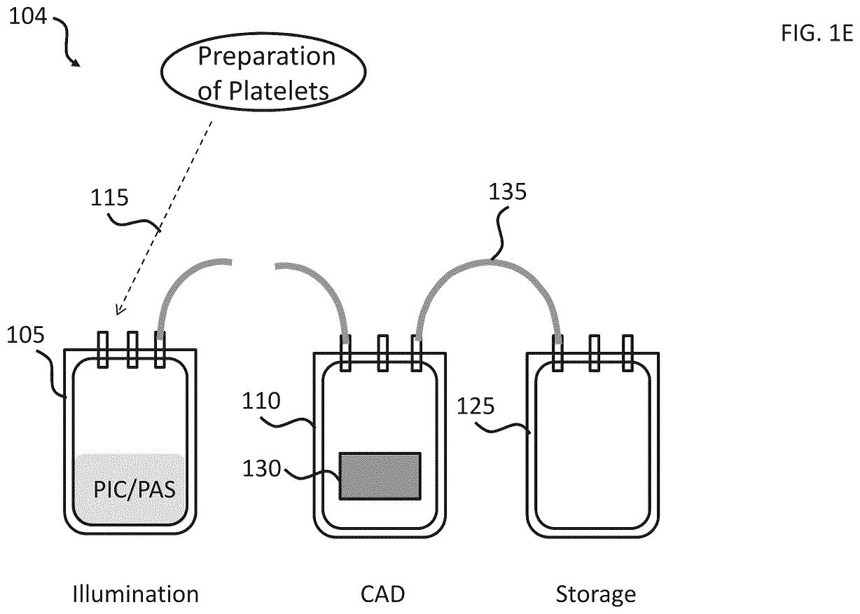

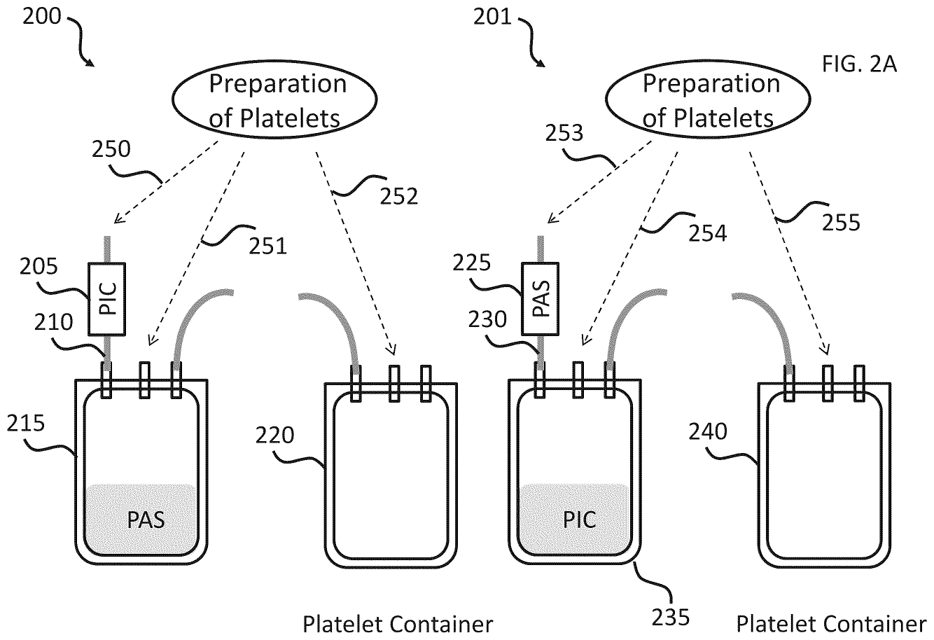

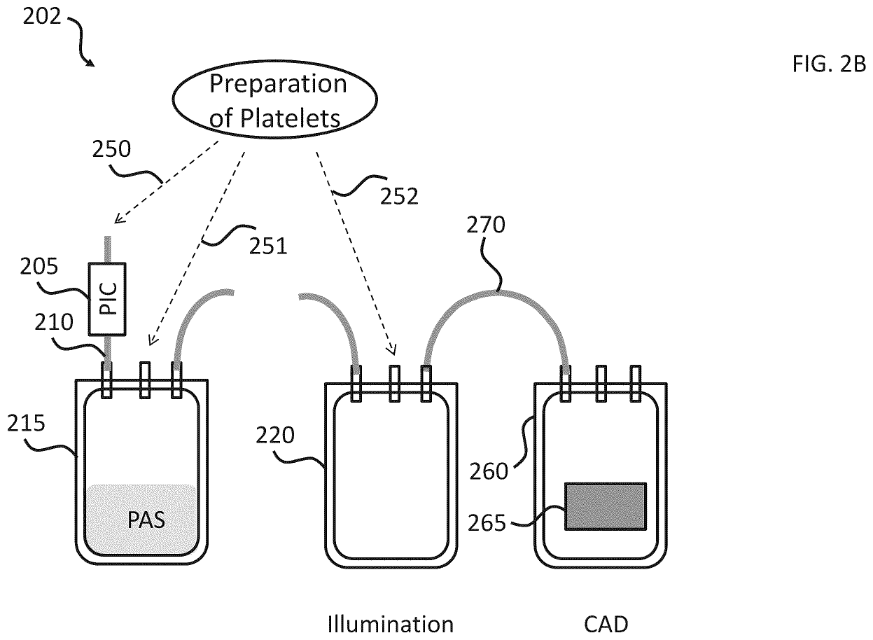

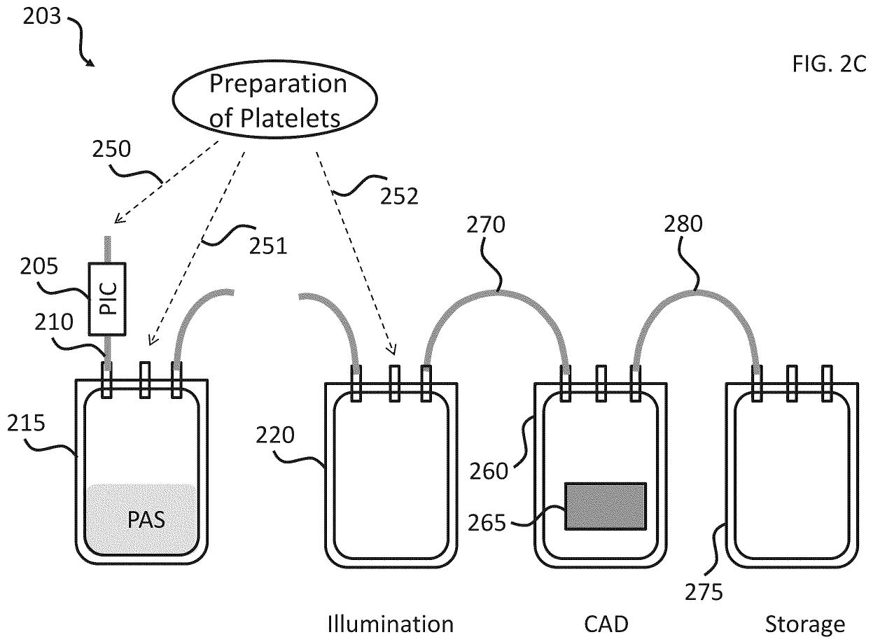

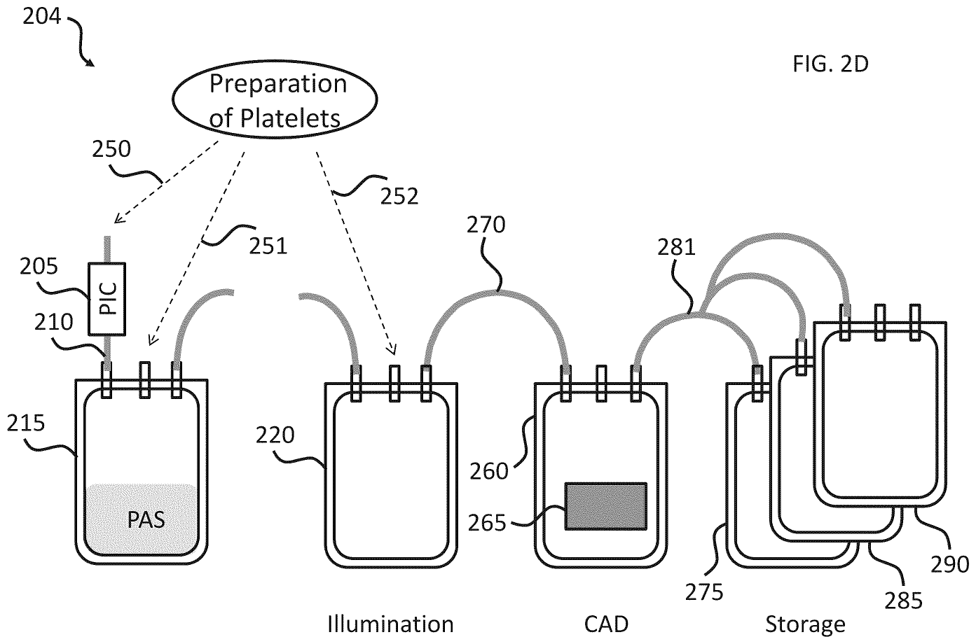

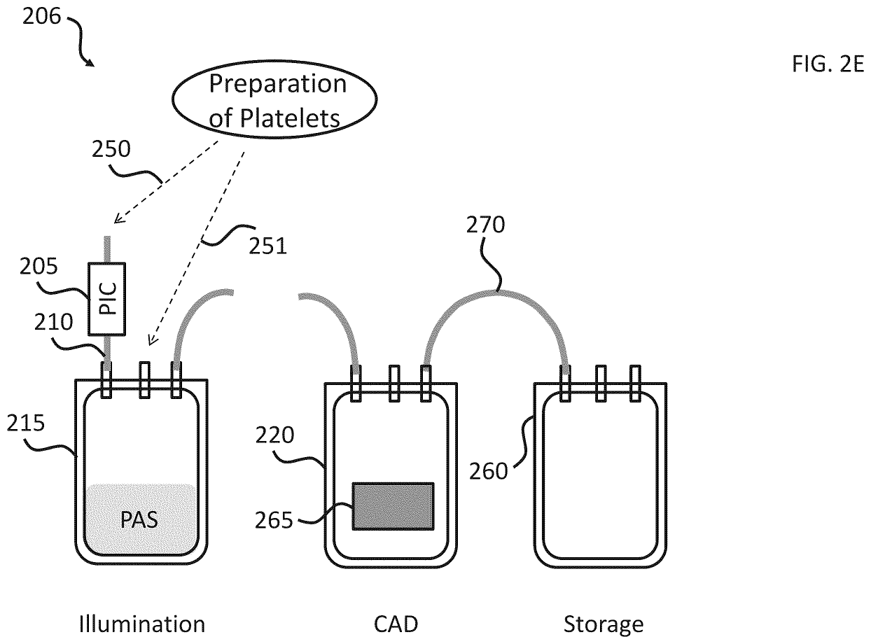

Non-limiting examples of kits for preparing a platelet composition according to the methods disclosed herein are illustrated in FIGS. 1A-1E and 2A-2E.

The exemplary kit 100 shown in FIG. 1A includes: (a) a first container 105 comprising a solution comprising a platelet additive solution (PAS) and a pathogen inactivation compound (PIC), and (b) a second container 110 (e.g., platelet container), wherein the first container 105 is not coupled to the second container 110. The dashed lines 115, 120 indicate that a preparation of platelets may be added to a first container 105 comprising a solution comprising a PAS and a PIC, or the preparation of platelets may be added to a second container 110. The first container 105 shown in FIG. 1A is suitable for admixing a preparation of platelets with a solution comprising a PAS and a PIC, and optionally is suitable for subjecting the preparation of platelets in admixture with the solution comprising the PAS and the PIC to light sufficient to photochemically inactivate a pathogen, if present. The second container 110 depicted in FIG. 1A is suitable for admixing a preparation of platelets with a solution comprising a PAS and a PIC and is suitable for containing the preparation of platelets in admixture with the solution comprising the PAS and the PIC. Furthermore, the second container 110 is suitable for one or more of: subjecting a preparation of platelets in admixture with a solution comprising a PAS and a PIC to light sufficient to photochemically inactivate a pathogen, if present; comprising a compound adsorption device (CAD); and storing a platelet composition. In some embodiments, the exemplary kit shown in FIG. 1A does not include a CAD.

An alternative configuration for an exemplary kit 101 of the disclosure is shown in FIG. 1B. This configuration optionally further includes a third container 125 coupled (e.g., via sterile tubing 135) to a second container 110, wherein the third container 125 comprises a compound adsorption device (CAD) 130. As depicted in FIG. 1B, the second container 110 is suitable for subjecting a preparation of platelets in admixture with a solution comprising a PAS and a PIC to light sufficient to photochemically inactivate a pathogen, if present. Furthermore, the third container 125 is optionally suitable for storing a platelet composition.

Another alternative configuration for an exemplary kit 102 of the disclosure is shown in FIG. 1C. This configuration optionally further includes a fourth container 140 that is suitable for storing a platelet composition, wherein a third container 125 comprising a compound adsorption device (CAD) 130 is coupled (e.g., via sterile tubing 145) to the fourth container 140.

Another alternative configuration for an exemplary kit 103 of the disclosure is shown in FIG. 1D. This configuration optionally further includes at least one storage container 140, 150, 155, wherein the at least one storage container 140, 150, 155 is suitable for storing a platelet composition, and wherein the at least one storage container 140, 150, 155 is coupled (e.g., via sterile tubing 146) to a third container 125 comprising a compound adsorption device (CAD) 130.

Another alternative configuration for an exemplary kit 104 of the disclosure is shown in FIG. 1E. As depicted in FIG. 1E, the first container 105 is suitable for admixing a preparation of platelets with a solution comprising a PAS and a PIC and further for subjecting a preparation of platelets in admixture with a solution comprising a PAS and a PIC to light sufficient to photochemically inactivate a pathogen, if present, and the second container 110 comprises a compound adsorption device (CAD) 130. This configuration further includes a third container 125 coupled (e.g., via sterile tubing 135) to a second container 110, wherein the third container 125 is optionally suitable for storing a platelet composition.

The exemplary kits 200, 201 shown in FIG. 2A include: (a) a first container comprising a platelet additive solution (PAS) 215, 225; (b) a second container comprising a pathogen inactivation compound (PIC) 205, 235; and (c) a third container (e.g., platelet container 220, 240) suitable for containing a preparation of platelets in admixture with the with the PAS and the PIC, wherein the first and second containers are coupled to one another (e.g., by a sealed but openable flow path 210, 230), and wherein neither of the first and second containers is coupled to the third container. The dashed lines 250, 251, 252, 253, 254, 255 indicate that a preparation of platelets may be added to a first container comprising a PAS, the preparation of platelets may be added to a second container comprising a PIC, or the preparation of platelets may be added to a third container. The first container shown in FIG. 2A (left side of page 200) is suitable for combining a PAS with a PIC, and further admixing a preparation of platelets. Alternatively, the second container shown in FIG. 2A (right side of page 201) is suitable for combining a PAS with a PIC, and further admixing a preparation of platelets, and optionally is suitable for subjecting the preparation of platelets in admixture with the solution comprising the PAS and the PIC to light sufficient to photochemically inactivate a pathogen, if present. Furthermore, in either configuration 200 or 201, the third container 220, 240 is suitable for one or more of: subjecting a preparation of platelets in admixture with a solution comprising a PAS and a PIC to light sufficient to photochemically inactivate a pathogen, if present; comprising a compound adsorption device (CAD); and storing a platelet composition. In some embodiments, the exemplary kits shown in FIG. 2A do not include a CAD.

An alternative configuration 202 for an exemplary kit of the disclosure is shown in FIG. 2B. This configuration optionally further includes a fourth container 265 coupled (e.g., via sterile tubing 270) to a third container 220, wherein the fourth container 265 comprises a compound adsorption device (CAD) 265. As depicted in FIG. 2B, the third container 220 is suitable for subjecting a preparation of platelets in admixture with a solution comprising a PAS and a PIC to light sufficient to photochemically inactivate a pathogen, if present. Furthermore, the fourth container 260 is optionally suitable for storing a platelet composition.

Another alternative configuration 203 for an exemplary kit of the disclosure is shown in FIG. 2C. This configuration optionally further includes a fifth container 275 that is suitable for storing a platelet composition, wherein a fourth container 260 comprising a compound adsorption device (CAD) 265 is coupled (e.g., via sterile tubing 280) to the fifth container 275.

Another alternative configuration 204 for an exemplary kit of the disclosure is shown in FIG. 2D. This configuration optionally further includes at least one storage container 275, 285, 290, wherein the at least one storage container 275, 285, 290 is suitable for storing a platelet composition, and wherein the at least one storage container 275, 285, 290 is coupled (e.g., via sterile tubing 281) to a fourth container 260 comprising a compound adsorption device (CAD) 265.

Another alternative configuration 206 for an exemplary kit of the disclosure is shown in FIG. 2E. As depicted in FIG. 2E, the first container 205 is suitable for combining a PAS with a PIC, and further admixing a preparation of platelets. The third container 220 comprises a compound adsorption device (CAD) 265. This configuration further includes a fourth container 260 coupled (e.g., via sterile tubing 270) to a third container 220, wherein the fourth container 260 is optionally suitable for storing a platelet composition.

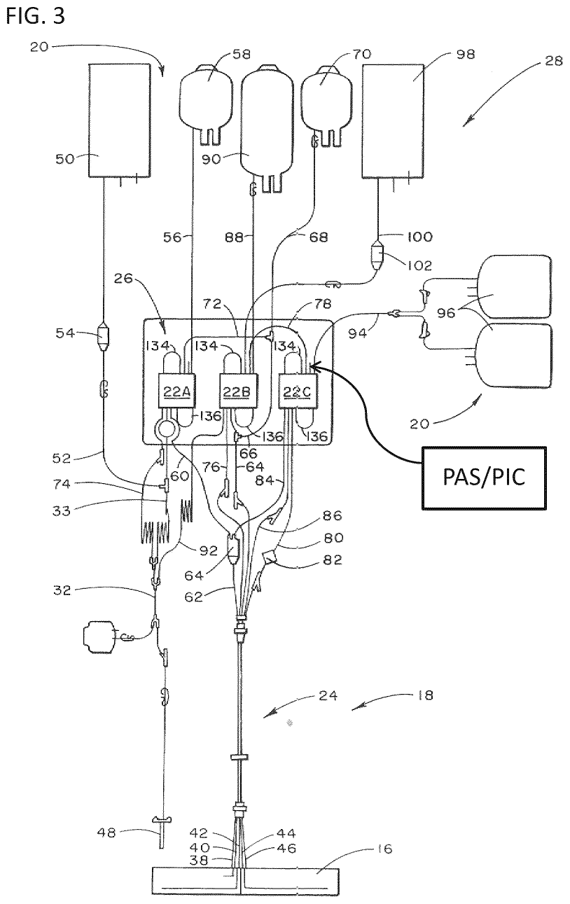

As disclosed herein, a preparation of platelets may be prepared by an apheresis method. As illustrated in FIG. 3, an apheresis device may be connected to any kit disclosed herein as the source of a preparation of platelets (e.g., platelets collected from a donor with the apheresis device), with a non-limiting example of a point for connection in the apheresis device depicted. The kits disclosed herein may be used with any apheresis device including those disclosed in U.S. Pat. No. 5,868,696. For example, as illustrated in FIGS. 1A-1E, an apheresis device may be connected to: a first container comprising a solution comprising a PAS and a PIC; and/or a second container. In other exemplary embodiments, as illustrated in FIGS. 2A-2E, an apheresis device may be connected to one or more of: a first container comprising a PAS; a second container comprising a PIC; and a third container.

### Compositions

The disclosure provides, in some aspects, compositions comprising a platelet additive solution (PAS) and a pathogen inactivation compound (PIC), wherein the composition is free of platelets.

In some embodiments of the composition comprising a PAS and a PIC, wherein the composition is free of platelets, the PAS comprises one or more of chloride, acetate, citrate, potassium, magnesium, phosphate, gluconate, glucose, and bicarbonate.

In some embodiments of the composition comprising a PAS and a PIC, wherein the composition is free of platelets, the PIC is a psoralen. In some embodiments of the composition comprising a PAS and a PIC, wherein the composition is free of platelets, the PIC is amotosalen. In some embodiments of the composition comprising a PAS and a PIC, wherein the composition is free of platelets, the PIC is selected from the group consisting of an isoalloxazine, an alloxazine, a phthalocyanine, a phenothiazine, a porphyrin, merocyanine 540, and salts or free bases thereof.

In some embodiments of the composition comprising a PAS and a PIC, wherein the composition is free of platelets, the solution comprising a PAS and a PIC has a volume of between about 100 mL and about 1000 mL. In some embodiments, the solution comprising a PAS and a PIC has a volume of between about 200 mL and about 900 mL, between about 300 mL and about 800 mL, between about 400 mL and about 700 mL, or between about 500 mL and about 600 mL. In some embodiments, the solution comprising a PAS and a PIC has a volume of about 100 mL, about 200 mL, about 300 mL, about 400 mL, about 500 mL, about 600 mL, about 700 mL, about 800 mL, about 900 mL, or about 1000 mL. In some embodiments, the solution comprising a PAS and a PIC has a volume of less than about 1000 mL, less than about 800 mL, less than about 600 mL, less than about 500 mL, less than about 400 mL, less than about 300 mL, or less than about 200 mL. In some embodiments, the solution comprising a PAS and a PIC has a volume of greater than about 800 mL, greater than about 700 mL, greater than about 600 mL, greater than about 500 mL, greater than about 400 mL, greater than about 300 mL, greater than about 200 mL, or greater than about 100 mL. In some embodiments, the solution comprising a PAS and a PIC has a volume of between about 1000 mL and about 5000 mL.

In some embodiments of the composition comprising a PAS and a PIC, wherein the composition is free of platelets, the concentration of PIC in the solution comprising a PAS and a PIC is about 25 μM to about 1200 μM, about 50 μM to about 1000 μM, about 50 μM to about 750 μM, about 50 μM to about 500 μM, about 75 μM to about 500 μM, about 100 μM to about 400 μM, about 150 μM to about 350 μM, about 200 μM to about 300 μM, or about 225 μM to about 250 μM. In some embodiments, the concentration of PIC in the solution comprising a PAS and a PIC is about 25 μM, about 50 μM, about 75 μM, about 100 μM, about 125 μM, about 150 PM, about 175 μM, about 200 μM, about 250 μM about 275 μM, about 300 μM, about 325 PM, about 350 PM, about 375 μM, about 400 μM, about 450 μM, about 500 μM, about 550 PM, about 600 PM, about 650 μM, about 700 μM, about 750 μM, about 800 μM, about 850 μM, about 900 PM, about 1000 μM, about 1100 μM, about 1200 μM, about 1300 μM, about 1400 μM, or about 1500 PM. In some embodiments, the concentration of PIC in the solution comprising a PAS and a PIC is about 225 μM to about 235 μM. In some embodiments, the concentration of PIC in the solution comprising a PAS and a PIC is about 225 μM, about 226 μM, about 227 μM, about 228 μM, about 229 PM, about 230 μM, about 231 μM, about 232 μM, about 233 μM, about 234 μM, or about 235 μM.

In some embodiments, the composition comprising a PAS and a PIC, wherein the composition is free of platelets, is a stock solution.

In some embodiments, the composition comprising a platelet additive solution (PAS) and a pathogen inactivation compound (PIC), wherein the composition is free of platelets, is sterile.

The present disclosure also provides, in some aspects, platelet compositions prepared by any of the methods described herein.

Disclosed examples and embodiments disclosed herein further describe methods, kits, and compositions for preparing a platelet composition suitable for infusion into an individual. The illustrated components and steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

“Comprising,” “having,” “containing,” and “including,” and other similar forms used herein are intended to be equivalent in meaning and be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. It is understood that “comprises” and grammatical equivalents thereof include “consisting of” or “consisting essentially of.”

Where a range of value is provided, it is understood that each intervening value, to the tenth of the unit of the lower limit unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein and in the appended claims, the singular forms “a,” “or,” and “the” include plural referents unless the context clearly dictates otherwise.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects. Rather, the scope of this disclosure should be determined with reference to the appended claims.

The disclosure is illustrated further by the following examples, which are not to be construed as limiting the disclosure in scope or spirit to the specific procedures described in them.

### EXAMPLES

### Example 1: Stability of Amotosalen in Platelet Additive Solution

Initial studies evaluated stability of the pathogen inactivation compound amotosalen (S-59) that was formulated in a platelet additive solution (PAS). A 230 μM solution of psoralen compound S-59 (Irsch et al., Transfus Med Hemother, 38: 19-31 (2011)) was prepared in the commercially available PAS solution InterSol® (Fenwal Inc.) and maintained at room temperature under ambient light conditions. HPLC analysis was performed on samples at times listed in Table 1 for both S-59 and photoproducts. The results shown in Table 1 demonstrate stability of S-59 in InterSol PAS over a 78 hour period in ambient light, with <6% loss of S-59 at 78 hours. S-59 data are shown as both concentration (μM), as well as peak area (S-59 UV) for relative comparison to any photoproducts detected with HPLC (Table 1). Peak D and Peak E photoproducts also were observed by 22 hr and 72 hr, respectively, as well as process impurity decomposition product 4′-HMT, with peak area values indicated (Table 1).

TABLE 1
HPLC analysis of S-59 and photoproducts.

| Sample | S-59 μM | S-59 UV | Peak D UV | Peak E UV | 4′-HMT UV |
|---|---|---|---|---|---|
| T 0 | 237.49 | 3700.439 | nd | nd | 4.924 |
| 5 hr | 230.62 | 3593.309 | nd | nd | 5.624 |
| 6 hr | 229.50 | 3575.825 | nd | nd | 5.090 |
| 22 hr | 223.71 | 3485.467 | 8.419 | nd | 5.125 |
| 30 hr | 226.56 | 3529.854 | 11.179 | nd | 5.334 |
| 47 hr | 223.76 | 3486.293 | 18.500 | nd | 4.954 |
| 56 hr | 220.82 | 3440.455 | 21.304 | nd | 4.742 |
| 72 hr | 226.88 | 3534.894 | 27.171 | 4.344 | 4.839 |
| 78 hr | 223.69 | 3485.093 | 28.960 | 4.623 | 4.793 |

Additional studies evaluated stability of the S-59 (amotosalen) pathogen inactivation compound formulated in a platelet additive solution (PAS), but protected from ambient light exposure. A 230 μM solution of S-59 was prepared in the commercially available PAS solution InterSol® (Fenwal Inc.) and maintained at room temperature protected from ambient light conditions. HPLC analysis was performed on samples at times listed in Table 2 for both S-59 and photoproducts. The results shown in Table 2 demonstrate stability of S-59 in InterSol PAS over a 77 hour period when protected from ambient light, with no loss of S-59 at 77 hours, shown as both concentration (μM) and peak area (UV). Peak D and Peak E photoproducts were not detected through 77 hours, while the process impurity decomposition product 4′-HMT was observed with peak area values indicated (Table 2).

TABLE 2
HPLC analysis of S-59 and photoproducts.

| Sample | S-59 μM | S-59 UV | 4′-HMT UV |
|---|---|---|---|
| T 0 | 228.82 | 3393.0647 | 3.8849 |
| 5.5 hr | 232.74 | 3451.6489 | 3.7162 |
| 21.5 hr | 231.93 | 3439.4907 | 4.3525 |
| 28.5 hr | 230.72 | 3421.447 | 3.8628 |
| 45.5 hr | 229.22 | 3399.0142 | 4.4036 |
| 53 hr | 230.72 | 3421.4934 | 4.0582 |
| 44 hr | 230.96 | 3425.1028 | 4.0199 |
| 77 hr | 232.41 | 3446.6414 | 4.4268 |

### Example 2: Stability of Amotosalen in 65% PAS with 35% Plasma and Platelets

A further study evaluated stability and photoconversion of amotosalen (S-59) in PAS with added plasma, and also containing platelets. S-59 was added at a concentration of 150 μM to 65% InterSol® PAS and 35% plasma a suspended preparation of platelets, and the admixture maintained in a platelet incubator during the study time course. Samples were removed for HPLC analysis at times 0, 5, 21, 29 and 48 hours, and the mixture was then treated with ˜3 J of UVA light at 55 hours with a post-UVA sample also analyzed by HPLC for both S-59 and photoproducts. The results shown in Table 3 demonstrate stability of S-59 in the PAS/plasma/platelets admixture, with no loss of S-59 prior to the UVA treatment at 55 hours, shown as both concentration (μM) and peak area (UV). Peak D and Peak E photoproducts were not detected (Table 3). UVA light treatment with the 55 hour samples demonstrated that photoconversion of S-59 was efficient after incubation of S-59 in PAS/plasma, with only 15.3% remaining in the post-UVA samples (Table 3).

TABLE 3
HPLC analysis of S-59 and photoproducts.

| Sample | S-59 μM | S-59 UV |
|---|---|---|
| T 0 | 148.12 | 1752.513 |
| 5 hr | 149.30 | 1738.331 |
| 21 hr | 147.81 | 1739.214 |
| 29 hr | 148.04 | 1725.882 |
| 48 hr | 148.06 | 1750.513 |
| 55 hr post-UVA | 22.71 | 274.897 |

### Example 3: Stability of Amotosalen in PAS/Plasma with Platelets and Bacteria

Another study evaluated 24 hour stability and photoconversion of amotosalen (5-59) in PAS with plasma and platelets, in the presence of bacteria. Two ABO matched platelet units in 100% plasma were pooled, split into two units and InterSol® PAS added (e.g., 65/35). S-59 was added to the test unit at a concentration of 150 μM, but not initially to the control unit. A log culture of K. pneumoniae (˜8 log cfu/mL) was added to the units at a target of ˜60 CFU/unit and the units were incubated in a platelets shaker at 22° C. for approximately 24 hours. At the end of incubation, S-59 was also added to the control unit at the same concentration and both control and test units were subjected to UVA light. Samples were taken pre- and post-UVA illumination for both units bacterial titer assay and HPLC. Post-illumination both units were subjected to a CAD processing step to remove residual amotosalen and photoproducts and stored on a platelet shaker for 7 days to confirm no growth of bacteria.

As shown in Table 4, S-59 concentration was stable for the 24 hour incubation period, with efficient photoconversion after UVA treatment at 24 hr, in both the control and test units. Thus the presence of K. pneumoniae did not adversely affect S-59 stability or photoconversion.

TABLE 4
Concentration of S-59 over 24 hour period and after UVA treatment.

| | Avg. | | Replicate 1 | | Replicate 2 | |
|---|---|---|---|---|---|---|
| | (S-59 μM) | | (S-59 μM) | | (S-59 μM) | |
| Time points | Control | Test | Control | Test | Control | Test |
| T_0 | 0 | 152 | 0 | 149 | 0 | 155 |
| T_24h | 149 | 153 | 144 | 149 | 153 | 157 |
| T_Post-UVA | 40 | 26 | 43 | 27 | 37 | 24 |
| % S-59 conversion | 27 | 17 | 30 | 18 | 24 | 15 |

Photochemical inactivation of K. pneumoniae was also measured post-UVA exposure, with the titer (log cfu/mL) results shown in the following table. High level inactivation was observed for both the control and test units, indicating no adverse impact on S-59 photochemical inactivation after 24 hours of storage in the admixture of PAS/Plasma and platelets with K. pneumoniae (Table 5).

TABLE 5
Inactivation of K. pneumoniae.

| | Replicate 1 | | Replicate 2 | |
|---|---|---|---|---|
| Unit ID | Control | Test | Control | Test |
| Titer 0 hour | 69-73 cfu/unit | | 86-101 cfu/unit | |
| Titer 24 hour | 6.6 | 6.5 | 5.6 | 5.4 |
| Titer Post-UVA | <−0.7 | <−0.7 | <−0.7 | <−0.7 |
| Log Reduction | >7.3 | >7.3 | >6.3 | >6.2 |

### Example 4: Inactivation of Calicivirus with Amotosalen in PAS/Plasma

Caliciviruses, such as feline calicivirus (FCV) which has been used as a model for hepatitis E virus, have previously been shown to be highly resistant to photochemical inactivation with amotosalen (S-59), with only about 1.7-2.4 log₁₀ reduction in titer (Irsch et al., Transfus Med Hemother, 38: 19-31 (2011)). From the results of Examples 1 and 2, showing that amotosalen is stable in PAS and PAS/Plasma, an additional study was performed that evaluated the level of inactivation of the calicivirus FCV with S-59 after extended incubation in PAS/Plasma. In this study, platelets in PAS/Plasma (65%/35%) were pooled to approximately 1320 mL, with approximately 16×10¹¹ total platelets, and spiked with a stock of the calicivirus FCV at a 1:100 dilution. A sample was taken from the FCV-spiked platelet pool to determine initial titer. The FCV-spiked platelets were then split into 5 units, at approximately 260 mL each, with approximately 3.2×10¹¹ platelets per unit. Each unit was dosed with S-59 at approximately 150 μM concentration, which is the concentration used commercially in the INTERCEPT® Blood System for pathogen inactivation treatment. Following the addition of S-59, the platelet units were incubated for 0, 2, 4, 8 or 24 hr. A control sample was taken at each time point for virus titer determination and HPLC analysis of S-59 concentration pre-UVA treatment, followed by UVA light exposure at ˜3 J/cm² to complete the photochemical treatment process for all test samples. Following UVA treatment, the control and test samples were evaluated for inactivation of FCV using a standard virological plaque assay. As shown in Table 6, incubation of the S-59/PAS/Plasma for 2 or more hours prior to UVA exposure resulted in dramatic increases in the level of FCV inactivation to below the limit of detection, as compared to the time 0 sample (no pre-incubation before UVA treatment). The data suggest a further advantage of the present disclosure, allowing for collection of platelets in a pre-mixed pathogen inactivation compound and additive solution (e.g., 5-59/PAS) and “pre-incubation” prior to the next UV exposure step in the photochemical treatment process (Table 6).

TABLE 6
FCV inactivation.

| | Titer (Log PFU/mL) | | Log |
|---|---|---|---|
| | 0J CONTROL | 3J TEST | Inactivation/mL |
| Stock | 8.1 | | n/a |
| FCV | 6.0 | | n/a |
| Pool | | | |
| T = 0 | 5.8 | 4.6 | 1.2 |
| T = 2 | 5.6 | <0.22* | >5.4 |
| T = 4 | 5.9 | <0.22* | >5.7 |
| T = 8 | 5.4 | <0.22* | >5.2 |
| T = 24 | 5.3 | <0.22* | >5.1 |

*Inactivated to the limit of detection.

Recalculations of the above inactivation data, normalized to the FCV pool titer, are shown in Table 7.

TABLE 7
Normalized FCV inactivation.

| | Titer (Log PFU/mL) | | Log |
|---|---|---|---|
| | 0J CONTROL | 3J TEST | Inactivation/mL |
| Stock | 8.1 | | n/a |
| FCV | 6.0 | | n/a |
| Pool | | | |
| T = 0 | 5.8 | 4.6 | 1.4 |
| T = 2 | 5.6 | <0.22* | >5.8 |
| T = 4 | 5.9 | <0.22* | >5.8 |
| T = 8 | 5.4 | <0.22* | >5.8 |
| T = 24 | 5.3 | <0.22* | >5.8 |

*Inactivated to the limit of detection.

An additional study was performed to evaluate the level of FCV inactivation with decreasing amounts of S-59 after extended incubation (e.g., pre-incubation) in PAS/Plasma. In this study, platelets in PAS/Plasma (65%/35%) were spiked with a stock of FCV at a 1:100 dilution. The FCV-spiked platelets were then split into 16 separate test units. Each unit was dosed with S-59 at approximately 150 μM, 90 μM, 30 μM or 15 μM concentration. Following the addition of S-59 in one of four dosing groups, the platelet units in each dosing group were incubated for 0, 4, 8 or 24 hr prior to illumination. A control sample was taken from each unit for virus titer determination and HPLC analysis of S-59 concentration pre-UVA treatment, followed by UVA light exposure at ˜3 J/cm² for photochemical treatment of the test samples. Following UVA treatment, the control and test samples were evaluated for inactivation of FCV using a standard virological plaque assay. Table 8 shows the FCV titers (log PFU/mL) for each sample and Table 9 shows the log inactivation for each sample.

TABLE 8
FCV Titers.

| | Control | 150 μM | 90 μM | 30 μM | 15 μM |
|---|---|---|---|---|---|
| 0 hr | 5.84 | 4.33 | 4.90 | 4.91 | 5.65 |
| 4 hr | 5.74 | <0.22 | <0.22 | 2.00 | 4.49 |
| 8 hr | 5.86 | <−0.48 | <−0.48 | <1.22 | 2.22 |
| 24 hr | 5.80 | <−0.48 | <−0.30 | <−0.22 | 0.52 |

TABLE 9
FCV Inactivation.

| | 150 μM | 90 μM | 30 μM | 15 μM |
|---|---|---|---|---|
| 0 hr | 1.51 | 0.94 | 0.93 | 0.19 |
| 4 hr | 5.62 | 5.62 | 3.84 | 1.35 |
| 8 hr | 5.84 | 5.84 | 4.62 | 3.62 |
| 24 hr | 5.84 | 5.84 | 5.62 | 5.32 |

As shown by these data, pre-incubation of the FCV containing platelets in S-59/PAS/Plasma prior to UVA illumination resulted in high levels of FCV inactivation, even with lower input concentrations of the S-59 pathogen inactivation compound. In particular, pre-incubation for 4, 8 or 24 hours in the case of both 150 μM and 90 μM S-59 concentration, 8 or 24 hours in the case of 30 μM S-59 concentration, or 24 hours in the case of 15 μM S-59 concentration, resulted in greater than 4 logs of FCV inactivation. Also, pre-incubation for 4 hr in the case of 30 μM S-59 concentration resulted in almost 4 logs FCV inactivation, and pre-incubation for 8 hr in the case of 15 μM S-59 concentration resulted in greater than 3.5 logs FCV inactivation. HPLC analysis also was performed to determine the amount (e.g., concentration) of S-59 remaining in samples after UVA illumination and photoconversion (Table 10).

TABLE 10
Post-UVA concentrations of S-59.

| | Post-UVA S-59 concentration (μM) | | | |
|---|---|---|---|---|
| Input S-59 | 0 hr | 4 hr | 8 hr | 24 hr |
| 150 μM | 29 | 33 | 22 | 19 |
| 90 μM | 14 | 10 | 11 | 9 |
| 30 μM | 5 | 2 | 2 | 3 |
| 15 μM | 3 | 2 | 2 | 2 |

As shown in Table 10, the residual S-59 concentrations post-treatment were reduced for all S-59 dosing groups with pre-incubation, including to levels less than 5 μM (e.g., 2 μM). These data indicate that pathogen inactivation treatment conditions can be achieved based on the methods provided herein, which result in high levels of inactivation (e.g., >4 logs) and also efficient S-59 photoconversion with residual S-59 concentrations of only 2 μM

### Example 5. Pathogen Inactivated Platelets Prepared with Pre-Mixed Amotosalen/PAS

Pathogen-inactivated platelets are prepared using kits and methods of the present disclosure. More specifically, in one example for preparation of a single unit, 3.9×10¹¹ platelets are collected from a donor in a volume of approximately 89.2 mL donor plasma (e.g., including any anti-coagulant) and transferred via sterilely connected tubing into a container of approximately 165.8 mL InterSol® PAS solution containing amotosalen at approximately 231 μM concentration (see e.g., FIG. 1C, 2C). The container with the admixture of platelets and amotosalen/PAS/Plasma with diluted (e.g., final) amotosalen concentration of approximately 150 μM is then sterile connected to the “dry side” remainder of a processing set (see e.g., FIG. 1C, 2C), and the admixture is transferred by gravity flow into the illumination container. Following treatment with approximately 3 J/cm² of UVA light using a commercially available INTERCEPT® Blood System illuminator (Cerus Corp.), the photochemically treated platelets are transferred to the CAD container for removal of residual amotosalen and photoproducts, and then transferred to a single storage container. In another example, pathogen-inactivated platelets are similarly prepared, but with 50% lower amotosalen concentrations (e.g., to yield an admixture of platelets and amotosalen/PAS/Plasma with diluted (e.g., final) amotosalen concentration of approximately 75 μM).

### Example 6. Pathogen Inactivated Platelets Prepared with Pre-Mixed Amotosalen/PAS

Pathogen-inactivated platelets are prepared using kits and methods of the present disclosure, whereby the amotosalen/PAS container(s) are directly connected to an Amicus© apheresis device (Fenwal Inc.). More specifically, in one example for preparation of two platelet units (e.g., double), a 500 mL container of InterSol® PAS solution with amotosalen added to a concentration of approximately 231 μM (see e.g., FIG. 1C) is sterilely connected to the apheresis device as depicted in FIG. 3. Approximately 7.6×10¹¹ platelets are collected by apheresis from the donor in a volume of approximately 178.5 mL donor plasma (e.g., including any anti-coagulant) and approximately 331.5 mL of the InterSol® PAS solution containing amotosalen is added automatically by the device to yield an admixture of platelets and amotosalen/PAS/Plasma with diluted (e.g., final) amotosalen concentration of approximately 150 μM. The platelet admixture is then transferred into two collection bags of the Amicus® device, with the platelets being distributed approximately evenly between the two bags. Next the two bags are disconnected from the apheresis device and each coupled separately by sterile connection to the “dry side” remainder of two separate processing sets (see e.g., FIG. 1C), and the admixture is transferred by gravity flow into the illumination container. Following treatment with approximately 3 J/cm² of UVA light using a commercially available INTERCEPT® Blood System illuminator (Cerus Corp.), the photochemically treated platelets are transferred to the CAD container for removal of residual amotosalen and photoproducts, and then each transferred to a single storage container, yielding two pathogen-inactivated platelet units. Alternatively, the collection bags removed from the apheresis device may be combined into a single illumination container by sterile connection to the “dry side” remainder of a processing set (see e.g., FIG. 1D), but with two final storage bags, and processed as described above, yielding two pathogen-inactivated platelet units. In another example, pathogen-inactivated platelets are similarly prepared, but with 50% lower amotosalen concentrations (e.g., to yield an admixture of platelets and amotosalen/PAS/Plasma with diluted (e.g., final) amotosalen concentration of approximately 75 μM).

### Exemplary Embodiments

Embodiment 1. A method of preparing a platelet composition, comprising:
   (a) providing in a first container a solution comprising a platelet additive solution (PAS) and a pathogen inactivation compound (PIC);
   (b) admixing the solution of step (a) with a preparation of platelets; and
   (c) subjecting the admixture of step (b) to light sufficient to photochemically inactivate a pathogen, if present, thereby yielding the platelet composition.
Embodiment 2. The method of embodiment 1, wherein the admixing of step (b) occurs in the first container.
Embodiment 3. The method of embodiment 1, wherein the admixing of step (b) occurs in a second container.
Embodiment 4. The method of embodiment 1 or embodiment 2, wherein the subjecting the admixture to light of step (c) occurs in the first container.
Embodiment 5. The method of any one of embodiments 1-3, wherein the subjecting the admixture to light of step (c) occurs in a second container.
Embodiment 6. The method of any one of embodiments 1-5, wherein the preparation of platelets is prepared by an apheresis method.
Embodiment 7. The method of embodiment 6, wherein the method further comprises, prior to step (b), connecting the first container to an apheresis device.
Embodiment 8. The method of embodiment 6 or embodiment 7, wherein the second container is connected to an apheresis device.
Embodiment 9. The method of any one of embodiments 1-5, wherein the preparation of platelets is prepared from one or more whole blood donation(s) by a buffy coat method or a platelet rich plasma (PRP) method.
Embodiment 10. The method of any one of embodiments 1-9, further comprising, after step (c): (d) transferring the platelet composition to a third container.
Embodiment 11. The method of embodiment 10, wherein the third container comprises a compound adsorption device (CAD).
Embodiment 12. The method of embodiment 10 or embodiment 11, wherein the third container is suitable for storage of the platelet composition.
Embodiment 13. The method of any one of embodiments 1-12, wherein the solution of step (a) comprises the PIC at a concentration of about 15 μM to about 1500 μM.
Embodiment 14. The method of any one of embodiments 1-13, wherein the PIC is a psoralen.
Embodiment 15. The method of embodiment 14, wherein the PIC is amotosalen.
Embodiment 16. The method of any one of embodiments 1-15, wherein the preparation of platelets comprises plasma, wherein the plasma comprises about 32 to 47% by volume of the admixture of step (b), with platelet additive solution comprising the remaining volume.
Embodiment 17. The method of embodiment 16, wherein the ratio of PAS to plasma by volume in the admixture of step (b) is about 65:35.
Embodiment 18. The method of any one of embodiments 1-17, wherein the admixture of step (b) comprises the PIC at a concentration sufficient to result in inactivation of at least 1 log of a pathogen, if present.
Embodiment 19. The method of any one of embodiments 1-18, wherein the admixture of step (b) comprises the PIC at a concentration sufficient to result in inactivation of at least 4 logs of a pathogen, if present.
Embodiment 20. The method of any one of embodiments 1-19, wherein the admixture of step (b) comprises the PIC at a concentration of about 5 μM to about 500 μM.
Embodiment 21. The method of embodiment 20, wherein the admixture of step (b) comprises the PIC at a concentration of about 145 μM to about 155 μM.
Embodiment 22. The method of embodiment 20, wherein the admixture of step (b) comprises the PIC at a concentration of about 30 μM to about 90 μM.
Embodiment 23. The method of any one of embodiments 1-22, wherein the PAS comprises one or more of chloride, acetate, citrate, potassium, magnesium, phosphate, gluconate, glucose, and bicarbonate.
Embodiment 24. The method of any one of embodiments 1-23, further comprising, prior to step (c):
   (b1) incubating the admixture of step (b) for a period of from 30 minutes to 24 hours.
Embodiment 25. The method of any one of embodiments 1-24, wherein the platelet composition comprises at least 2×10¹¹ platelets.
Embodiment 26. The method of any one of embodiments 1-25, wherein the method is sufficient to inactivate at least 1 log of a pathogen, if present, and wherein the platelet composition after step (c) is suitable for infusion into a subject without further processing to remove residual PIC or photoproducts thereof.
Embodiment 27. The method of any one of embodiments 1-26, wherein the method is sufficient to inactivate at least 1 log of a pathogen, if present, and wherein the platelet composition after step (c) is suitable for infusion into a subject without transferring the platelet composition to a container comprising a compound adsorption device (CAD).
Embodiment 28. The method of any one of embodiments 1-27, wherein the method is sufficient to inactivate at least 1 log of a pathogen, if present, and wherein the platelet composition after step (c) comprises 5 μM or less of PIC.
Embodiment 29. The method of any one of embodiments 1-28, wherein the method is sufficient to inactivate at least 4 log of the pathogen, if present, wherein the platelet composition after step (c) comprises 2 μM or less of PIC, and wherein the concentration of PIC in the admixture of the preparation of platelets and the solution comprising PAS and PIC is about 15 μM to about 150 μM.
Embodiment 30. A method of preparing a platelet composition, comprising (a) providing a solution comprising a platelet additive solution (PAS) and a pathogen inactivation compound (PIC); (b) admixing the solution of step (a) with a preparation of platelets; (c) incubating the admixture of a preparation of platelets and a solution comprising a PAS and a PIC for a period of about 30 minutes to about 24 hours; and (d) subjecting the incubated admixture of step (c) to light sufficient to photochemically inactivate a pathogen, if present, thereby yielding the platelet composition, wherein:
   (i) the method is sufficient to inactivate at least 1 log of a pathogen, if present;
   (ii) the concentration of PIC in the admixture of the preparation of platelets and the solution comprising PAS and PIC is about 15 μM to about 150 μM; and
   (iii) the platelet composition after subjecting the admixture of the preparation of platelets and the solution comprising PAS and PIC to light comprises less than 5 μM of PIC.
Embodiment 31. A kit for preparing a platelet composition, comprising:
   (a) a first container comprising a solution comprising a platelet additive solution (PAS) and a pathogen inactivation compound (PIC), and
   (b) a second container suitable for containing a preparation of platelets in admixture with the solution comprising the PAS and the PIC,
   wherein the first container is not coupled to the second container.
Embodiment 32. The kit of embodiment 31, wherein the first container is suitable for admixing the preparation of platelets with the solution comprising the PAS and the PIC.
Embodiment 33. The kit of embodiment 31 or embodiment 32, wherein the second container is suitable for admixing the preparation of platelets with the solution comprising the PAS and the PIC.
Embodiment 34. The kit of any one of embodiments 31-33, wherein the second container is suitable for subjecting the preparation of platelets in admixture with the solution comprising the PAS and the PIC to light sufficient to photochemically inactivate a pathogen, if present.
Embodiment 35. The kit of any one of embodiments 31-34, wherein the first container is suitable for subjecting the preparation of platelets in admixture with the solution comprising the PAS and the PIC to light sufficient to photochemically inactivate a pathogen, if present.
Embodiment 36. The kit of any one of embodiments 31-35, wherein the second container comprises a compound adsorption device (CAD).
Embodiment 37. The kit of any one of embodiments 31-36, wherein the second container is suitable for storing the platelet composition.
Embodiment 38. The kit of any one of embodiments 31-37, further comprising a third container, wherein the third container comprises a compound adsorption device (CAD), and wherein the third container is coupled to the second container.
Embodiment 39. The kit of any one of embodiments 31-38, further comprising at least one storage container, wherein the at least one storage container is suitable for storing the platelet composition, and wherein the at least one storage container is coupled to the second container or to the third container, if present.
Embodiment 40. The kit of any one of embodiments 31-39, wherein the solution comprising the PAS and the PIC has a volume of between about 100 mL and about 1000 mL.
Embodiment 41. The kit of any one of embodiments 31-40, wherein the PIC is at a concentration of about 15 μM to about 1500 μM.
Embodiment 42. The kit of any one of embodiments 31-41, wherein the PIC is a psoralen.
Embodiment 43. The kit of embodiment 42, wherein the PIC is amotosalen.
Embodiment 44. The kit of any one of embodiments 31-43, wherein the first container, the second container, or both the first container and second container is suitable for connecting to an apheresis device or to a container containing a preparation of platelets.
Embodiment 45. A kit for preparing a platelet composition, comprising:
   (a) a first container comprising a platelet additive solution (PAS);
   (b) a second container comprising a pathogen inactivation compound (PIC); and
   (c) a third container suitable for containing a preparation of platelets in admixture with the with the PAS and the PIC,
   wherein the first and second containers are coupled to one another, and wherein neither of the first and second containers is coupled to the third container.
Embodiment 46. The kit of embodiment 45, wherein the second container is suitable for combining the PAS with the PIC.
Embodiment 47. The kit of embodiment 45, wherein the first container is suitable for combining the PAS with the PIC.
Embodiment 48. The kit of any one of embodiments 45-47, wherein the second container is suitable for admixing the preparation of platelets with the PAS and the PIC.
Embodiment 49. The kit of any one of embodiments 45-47, wherein the first container is suitable for admixing the preparation of platelets with the PAS and the PIC.
Embodiment 50. The kit of any one of embodiments 45-47, wherein the third container is suitable for admixing the preparation of platelets with the PAS and the PIC.
Embodiment 51. The kit of any one of embodiments 45-50, wherein the third container is suitable for subjecting the preparation of platelets in admixture with the PAS and the PIC to light sufficient to photochemically inactivate a pathogen, if present.
Embodiment 52. The kit of any one of embodiments 45-50, wherein the second container is suitable for subjecting the preparation of platelets in admixture with the PAS and the PIC to light sufficient to photochemically inactivate a pathogen, if present.
Embodiment 53. The kit of any one of embodiments 45-50, wherein the first container is suitable for subjecting the preparation of platelets in admixture with the PAS and the PIC to light sufficient to photochemically inactivate a pathogen, if present.
Embodiment 54. The kit of any one of embodiments 45-53, wherein the third container comprises a compound adsorption device (CAD).
Embodiment 55. The kit of any one of embodiments 45-54, wherein the third container is suitable for storing the platelet composition.
Embodiment 56. The kit of any one of embodiments 45-55, further comprising a fourth container, wherein the fourth container comprises a compound adsorption device (CAD), and wherein the fourth container is coupled to the third container.
Embodiment 57. The kit of any one of embodiments 45-56, further comprising at least one storage container, wherein the at least one storage container is suitable for storing the platelet composition, and wherein the at least one storage container is coupled to the third container or to the fourth container, if present.
Embodiment 58. The kit of any one of embodiments 45-57, wherein the PIC is a psoralen.
Embodiment 59. The kit of embodiment 58, wherein the PIC is amotosalen.
Embodiment 60. The kit of any one of embodiments 45-59, wherein the first container, the second container, or both the first container and second container is suitable for connecting to an apheresis device or to a container containing a preparation of platelets.
Embodiment 61. A composition comprising a pathogen inactivation compound (PIC) and a platelet additive solution (PAS), wherein the composition is free of platelets.
Embodiment 62. The composition of embodiment 61, wherein the concentration of the PIC is about 15 μM to about 1500 μM.
Embodiment 63. The composition of embodiment 61 or embodiment 62, wherein the PIC is a psoralen.
Embodiment 64. The composition of embodiment 63, wherein the PIC is amotosalen.
Embodiment 65. The composition of any one of embodiments 61-64, wherein the PAS comprises one or more of chloride, acetate, citrate, potassium, magnesium, phosphate, gluconate, glucose, and bicarbonate.
Embodiment 66. The composition of any one of embodiments 61-65, wherein the composition is sterile.
Embodiment 67. A platelet composition prepared by the method of any one of embodiments 1-30.

1-67. (canceled)

68. A method of preparing a platelet composition, comprising:
(a) providing:
(i) a first container containing a first volume of a solution comprising a platelet additive solution (PAS) and a pathogen inactivation compound (PIC) and
(ii) a second container containing a second volume of a solution comprising a PAS and a PIC, wherein the first and second volumes are different;
(b) selecting the first or the second container for use in preparing the platelet composition;
(c) admixing the solution of the selected container with a preparation of platelets; and
(d) subjecting the admixture of step (c) to light sufficient to photochemically inactivate a pathogen, if present, thereby yielding the platelet composition.

69. The method of claim 68, wherein the first or second container is selected for use based at least in part on a volume of the preparation of platelets, an amount of platelets in the preparation of platelets, or number of platelet compositions to be prepared.

70. The method of claim 68, wherein the first volume is sufficient for preparing one therapeutic dose of platelets.

71. The method of claim 68, wherein the second volume is sufficient for preparing two therapeutic doses of platelets.

72. The method of claim 68, wherein step (a) further comprises providing a third container containing a third volume of a solution comprising a PAS and a PIC, wherein the first, second, and third volumes are different.

73. The method of claim 72, wherein the third volume is sufficient for preparing three therapeutic doses of platelets.

74. The method of claim 68, wherein the admixing of step (c) occurs in the selected first or second container.

75. The method of claim 68, wherein the admixing of step (c) occurs in a third container.

76. The method of claim 68, wherein subjecting the admixture to light of step (d) occurs in the selected first or second container.

77. The method of claim 68, wherein subjecting the admixture to light of step (d) occurs in a third container.

78. The method of claim 68, further comprising, after step (d), transferring the platelet composition to a container comprising a compound adsorption device (CAD).

79. The method of claim 68, further comprising, after step (d), transferring the platelet composition to a storage container suitable for storing the platelet composition.

80. The method of claim 68, wherein the PIC is a psoralen.

81. The method of claim 80, wherein the PIC is amotosalen.

82. The method of claim 68, wherein the admixture of step (c) comprises the PIC at a concentration of about 5 μM to about 500 μM.

83. The method of claim 68, wherein:
(i) the method is sufficient to inactivate at least 1 log of a pathogen, if present;
(ii) the concentration of PIC in the admixture of the preparation of platelets and the solution comprising PAS and PIC is about 15 μM to about 150 μM; and
(iii) the platelet composition after subjecting the admixture of the preparation of platelets and the solution comprising PAS and PIC to light comprises less than 5 μM of PIC.

84. The method of claim 68, wherein the method is sufficient to inactivate at least 1 log of a pathogen, if present, and wherein the platelet composition after step (d) is suitable for infusion into a subject without further processing to remove residual PIC or photoproducts thereof.

85. A method of preparing a platelet composition, comprising:
(a) providing in a first container a solution comprising a platelet additive solution (PAS) and a pathogen inactivation compound (PIC);
(b) connecting the first container to an apheresis device;
(c) admixing the solution of step (a) with a preparation of platelets prepared with the apheresis device; and
(d) subjecting the admixture of step (c) to light sufficient to photochemically inactivate a pathogen, if present, thereby yielding the platelet composition.

86. The method of claim 85, wherein the admixing of step (c) occurs in the first container.

87. The method of claim 85, wherein the admixing of step (c) occurs in a second container connected to the apheresis device.

88. The method of claim 85, wherein subjecting the admixture to light of step (d) occurs in the first container.

89. The method of claim 85, wherein subjecting the admixture to light of step (d) occurs in a second container.

90. The method of claim 85, further comprising, after step (d):
(e) transferring the platelet composition to a third container.

91. The method of claim 90, wherein the third container comprises a compound adsorption device (CAD).

92. The method of claim 90, wherein the third container is suitable for storage of the platelet composition.

93. The method of claim 85, wherein the PIC is a psoralen.

94. The method of claim 93, wherein the PIC is amotosalen.

95. The method of claim 85, wherein the admixture of step (c) comprises the PIC at a concentration of about 5 μM to about 500 μM.

96. The method of claim 85, wherein:
(i) the method is sufficient to inactivate at least 1 log of a pathogen, if present;
(ii) the concentration of PIC in the admixture of the preparation of platelets and the solution comprising PAS and PIC is about 15 μM to about 150 μM; and
(iii) the platelet composition after subjecting the admixture of the preparation of platelets and the solution comprising PAS and PIC to light comprises less than 5 μM of PIC.

97. A kit for preparing a platelet composition, comprising:
(a) a first container comprising a solution comprising a platelet additive solution (PAS) and a pathogen inactivation compound (PIC), and
(b) a second container suitable for containing a preparation of platelets in admixture with the solution comprising the PAS and the PIC,
wherein the first container is not coupled to the second container.

98. The kit of claim 97, comprising two or more first containers, wherein each of the two or more first containers contains a different volume of a solution comprising a PAS and a PIC.

99. The kit of claim 97, wherein the first container is suitable for admixing the preparation of platelets with the solution comprising the PAS and the PIC.

100. The kit of claim 97, wherein the second container is suitable for admixing the preparation of platelets with the solution comprising the PAS and the PIC.

101. The kit of claim 97, wherein the second container comprises a compound adsorption device (CAD).

102. The kit of claim 97, wherein the second container is suitable for storing the platelet composition.

103. The kit of claim 97, further comprising a third container, wherein the third container comprises a compound adsorption device (CAD), and wherein the third container is coupled to the second container.

104. The kit of claim 97, further comprising at least one storage container, wherein the at least one storage container is suitable for storing the platelet composition, and wherein the at least one storage container is coupled to the second container or to a third container, if present.

105. The kit of claim 97, wherein the first container, the second container, or both the first container and second container is suitable for connecting to an apheresis device or to a container containing a preparation of platelets.

106. The kit of claim 97, wherein the PIC is a psoralen.

107. A kit for preparing a platelet composition, comprising:
(a) a first container comprising a platelet additive solution (PAS);
(b) a second container comprising a pathogen inactivation compound (PIC); and
(c) a third container suitable for containing a preparation of platelets in admixture with the with the PAS and the PIC,
wherein the first and second containers are coupled to one another, and wherein neither of the first and second containers is coupled to the third container.

108. The kit of claim 107, wherein the second container is suitable for combining the PAS with the PIC.

109. The kit of claim 107, wherein the first container is suitable for combining the PAS with the PIC.

110. The kit of claim 107, wherein the second container is suitable for admixing the preparation of platelets with the PAS and the PIC.

111. The kit of claim 107, wherein the first container is suitable for admixing the preparation of platelets with the PAS and the PIC.

112. The kit of claim 107, wherein the third container is suitable for admixing the preparation of platelets with the PAS and the PIC.

113. The kit of claim 107, wherein the third container is suitable for subjecting the preparation of platelets in admixture with the PAS and the PIC to light sufficient to photochemically inactivate a pathogen, if present.

114. The kit of claim 107, wherein the second container is suitable for subjecting the preparation of platelets in admixture with the PAS and the PIC to light sufficient to photochemically inactivate a pathogen, if present.

115. The kit of claim 107, wherein the first container is suitable for subjecting the preparation of platelets in admixture with the PAS and the PIC to light sufficient to photochemically inactivate a pathogen, if present.

116. The kit of claim 107, wherein the third container comprises a compound adsorption device (CAD).

117. The kit of claim 107, wherein the third container is suitable for storing the platelet composition.

118. The kit of claim 107, further comprising a fourth container, wherein the fourth container comprises a compound adsorption device (CAD), and wherein the fourth container is coupled to the third container.

119. The kit of claim 107, further comprising at least one storage container, wherein the at least one storage container is suitable for storing the platelet composition, and wherein the at least one storage container is coupled to the third container or to a fourth container, if present.
